(19) United States
(12) Patent Application Publication
(10) Pub. No.: US 2025/0197904 A1
(43) Pub. Date: Jun. 19, 2025

(54) A PROCESS FOR THE SYNTHESIS OF ( R)-2-(( TERT-BUTOXYCARBONYL)AMINO)-3-(DIETHOXYPHOSPHORYL)PROPANOIC ACID OR OF PHOSPHONATE DERIVATIVES THEREOF

(71) Applicant: VIATRIS ASIA PACIFIC PTE. LTD., Singapore (SG)

(72) Inventors: Stefan Abele, Allschwil (CH); Gareth Brown, Antrim (GB); Peter McIntyre, Belfast (GB); Stefan Mix, Belfast (GB)

(21) Appl. No.: 18/844,771

(22) PCT Filed: Mar. 10, 2023
(86) PCT No.: PCT/EP2023/056138

§ 371 Date: Sep. 6, 2024

(30) Foreign Application Priority Data
Mar. 14, 2022 (WO) PCT/EP2022/056471

(51) Int. Cl. C12P 17/16; C12P 13/00

(52) U.S. Cl. CPC C12P 17/16; C12P 13/005

(57) ABSTRACT

The present invention relates to a process for the synthesis of (R)-2-((tert-butoxycarbonyl)amino)-3-(diethoxyphosphoryl)propanoic acid (“COMPOUND”), of phosphonate derivatives thereof, or of salts of any of the aforementioned; to a crystalline form of COMPOUND, and to the use of COMPOUND (especially of COMPOUND in crystalline form) or phosphonate derivatives or salts thereof for the preparation of 4-((R)-2-{[6-((S)-3-methoxy-pyrrolidin-1-yl)-2-phenyl-pyrimidine-4-carbonyl]-amino}-3-phosphono-propionyl)-piperazine-1-carboxylic acid butyl ester (also known as selatogrel), or of a pharmaceutically acceptable salt thereof.

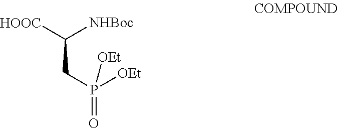 [SMILES: CCOP(=O)(C[C@H](NC(=O)OC(C)(C)C)C(=O)O)OCC]

The present invention relates to a process for the synthesis of (R)-2-((tert-butoxycarbonyl)amino)-3-(diethoxyphosphoryl)propanoic acid (hereinafter also referred to as “COMPOUND”), of phosphonate derivatives thereof, or of salts of any of the aforementioned; to a crystalline form of COMPOUND, and to the use of COMPOUND (especially of COMPOUND in crystalline form) or phosphonate derivatives or salts thereof for the preparation of 4-((R)-2-{[6-((S)-3-methoxy-pyrrolidin-1-yl)-2-phenyl-pyrimidine-4-carbonyl]-amino}-3-phosphono-propionyl)-piperazine-1-carboxylic acid butyl ester (also known as selatogrel), or of a pharmaceutically acceptable salt thereof.

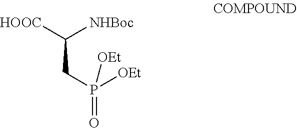 [SMILES: CCOP(=O)(C[C@H](NC(=O)OC(C)(C)C)C(=O)O)OCC]

The preparation of selatogrel from COMPOUND is described in WO 2009/069100 and Caroff E et al., J. Med. Chem. (2015), 58, 9133-9153. The medical use of selatogrel is for instance described in WO 2018/167139; Baldoni D et al., Clin Drug Investig (2014), 34 (11), 807-818; Storey R F et al., European Heart Journal, ehz807, doi:10.1093/eurheartj/ehz807; and Sinnaeve P R et al, J Am Coll Cardiol (2020), 75 (20), 2588-97 (doi.org/10.1016/j.jacc.2020.03.059). Selatogrel is investigated in a multi-center, double-blind, randomized, placebo-controlled, parallel-group study to evaluate the efficacy and safety of self-administered subcutaneous selatogrel for prevention of all-cause death and treatment of acute myocardial infarction in subjects with a recent history of acute myocardial infarction (ClinicalTrials.gov Identifier: NCT04957719).

For instance, COMPOUND can be prepared starting from commercially available methyl (R)-2-((tert-butoxycarbonyl)amino)-3-iodopropanoate (Boc-3-iodo-L-Ala-OMe) by reaction with triethylphosphite and subsequent saponification with LiOH in a mixture of water and THF (WO 2009/069100 and Caroff E et al., J. Med. Chem. (2015), 58, 9133-9153). This process has the disadvantage of using the highly expensive starting material Boc-3-iodo-L-Ala-OMe and its tendency to racemize under conditions of the Arbuzov reaction, especially in large scale synthesis. Alternatively, COMPOUND as a racemic mixture together with its enantiomer can be prepared according to the procedure shown in Scheme 1: Compound 2 can be prepared by reaction of ethyl 3-bromo-2-oxopropanoate with hydroxylamine hydrochloride. By reaction of compound 2 with triethylphosphite ethyl 3-(diethoxyphosphoryl)-2-(hydroxyimino)-propanoate (compound 3) can be obtained which can be transformed to compound 4 by hydrogenation in the presence of Pd/C as a catalyst and protection of the amino group with Boc₂O. A racemic mixture of COMPOUND and its enantiomer can be obtained from compound 4 by saponification with LiOH in a mixture of water and toluene at a temperature of about 20° C. (Stefan Abele, presentation at 250^{th} ACS national meeting, Aug. 17, 2015, Boston; Stefan Abele, presentation at Swiss Industrial Chemistry Symposium Oct. 28, 2016, Basel). Even if a separation of enantiomers by chiral column chromatography or simulated moving bed chromatography at the stage of the ester (compound 4), the final acid (racemate of COMPOUND) or a later intermediate in the synthesis of compound 5 (such as for instance 4-[2-tert-butoxycarbonylamino-3-(diethoxy-phosphoryl)-propionyl]-piperazine-1-carboxylic acid butyl ester) may be possible, such separation of enantiomers has the disadvantages of limited scaleability and throughput and the need for huge amounts of solvent.

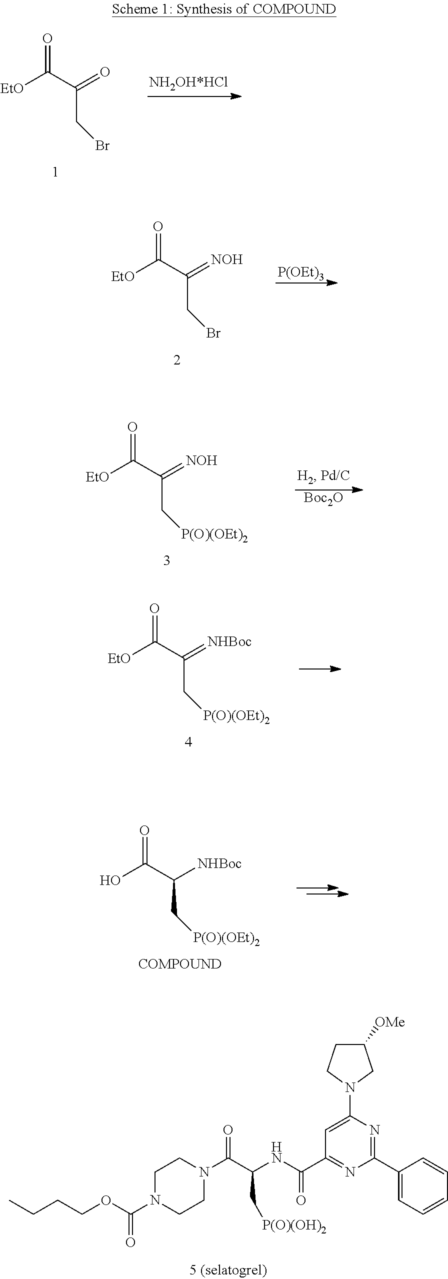

Surprisingly, it was found that COMPOUND could be obtained on large scale in excellent yield and high enantiomeric purity by enzymatic resolution with hydrolases.

### DESCRIPTION OF THE FIGURES

FIG. 1 shows the X-ray powder diffraction diagram of COMPOUND in the crystalline form (I), wherein the X-ray powder diffraction diagram was measured with the XRPD method described in the experimental part and is displayed against Cu Kα radiation. The X-ray diffraction diagram shows peaks having a relative intensity, as compared to the most intense peak in the diagram, of the following percentages (relative peak intensities given in parenthesis) at the indicated angles of refraction 2 theta (selected peaks from the range 3-30° 2 theta with relative intensity larger than 10% are reported): 9.8° (100%), 10.3° (37%), 12.5° (12%), 13.3° (16%), 16.2° (23%), 17.5° (34%), 19.8° (14%), 20.7° (24%), 22.4° (25%), 22.7° (15%), 24.2° (23%), 24.7° (14%), 26.8° (12%), and 27.3° (33%).

In the X-ray diffraction diagram of FIG. 1 the angle of refraction 2 theta (20) is plotted on the horizontal axis and the counts on the vertical axis.

For avoidance of any doubt, the above-listed peaks describe the experimental results of the X-ray powder diffraction shown in FIG. 1. It is understood that, in contrast to the above peak list, only a selection of characteristic peaks is required to fully and unambiguously characterize COMPOUND in the respective crystalline form of the present invention.

FIG. 2 shows the gravimetric vapour sorption (GVS) diagram of COMPOUND in the crystalline form (I) at 25° C. as obtained from example 4.

In the gravimetric vapour sorption diagram of FIG. 2 the relative humidity (% RH) is plotted on the horizontal axis and the mass change (% dm) on the vertical axis.

FIG. 3 shows the differential scanning calorimetry (DSC) thermogram of COMPOUND in the crystalline form (I). In the DSC thermogram of FIG. 3 the temperature (C) is plotted on the horizontal axis and the heat flow (mW) on the vertical axis.

FIG. 4 shows the X-ray powder diffraction diagram of ethyl 3-(diethoxyphosphoryl)-2-(hydroxyimino)propanoate in the crystalline form (A), wherein the X-ray powder diffraction diagram was measured with the XRPD method described in the experimental part and is displayed against Cu Kα radiation. The X-ray diffraction diagram shows peaks having a relative intensity, as compared to the most intense peak in the diagram, of the following percentages (relative peak intensities given in parenthesis) at the indicated angles of refraction 2 theta (selected peaks from the range 3°-40° 2 theta with relative intensity larger than 8% are reported): 10.2° (100%), 11.3° (99%), 13.2° (11%), 13.8° (9%), 15.2° (9%), 20.5° (13%), 22.7° (71%), 22.9° (33%), 25.3° (11%), 26.6° (18%), and 34.3° (49%).

FIG. 5 shows the X-ray powder diffraction diagram of ethyl 3-(diethoxyphosphoryl)-2-(hydroxyimino)propanoate in the crystalline form (B), wherein the X-ray powder diffraction diagram was measured with the XRPD method described in the experimental part and is displayed against Cu Kα radiation. The X-ray diffraction diagram shows peaks having a relative intensity, as compared to the most intense peak in the diagram, of the following percentages (relative peak intensities given in parenthesis) at the indicated angles of refraction 2 theta (selected peaks from the range 3°-40° 2 theta with relative intensity larger than 10% are reported): 10.9° (31%), 18.2° (23%), 19.5° (100%), 19.8° (24%), 25.3° (10%), 27.2° (91%), 28.7° (11%), 29.5° (65%), 33.0° (24%), 34.7° (22%), and 36.9° (30%). In the X-ray diffraction diagrams of FIG. 4 and FIG. 5, respectively, the angle of refraction 2 theta (2θ) is plotted on the horizontal axis and the counts on the vertical axis.

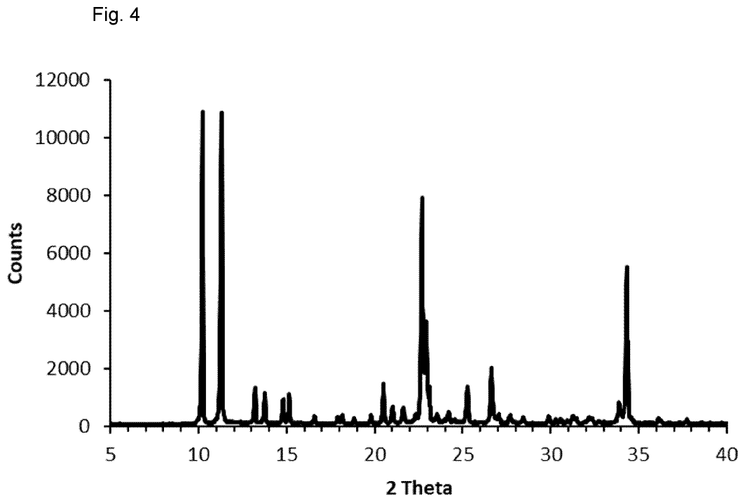

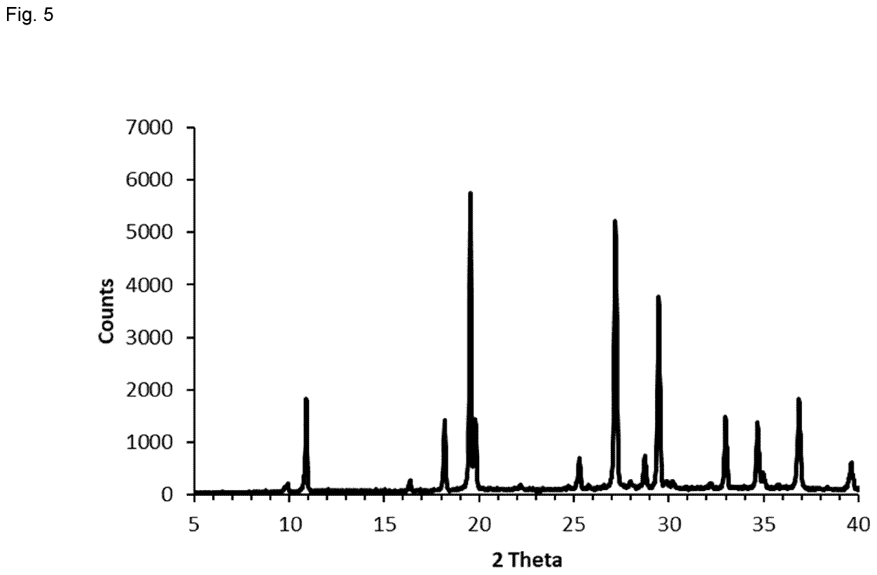

For avoidance of any doubt, the above-listed peaks describe the experimental results of the X-ray powder diffraction shown in FIGS. 4 and 5, respectively. It is understood that, in contrast to the above peak list, only a selection of characteristic peaks is required to fully and unambiguously characterize ethyl 3-(diethoxyphosphoryl)-2-(hydroxyimino)propanoate in the respective crystalline form of the present invention.

### DESCRIPTION OF THE INVENTION

In the following the present invention will be described and various embodiments of the invention are presented.

1) In a first embodiment, the present invention relates to a process for the manufacturing of a compound of formula (I), or of a salt thereof,

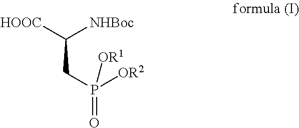

said process comprising the step of reacting a compound of formula (II)

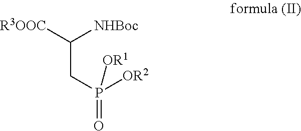

wherein
R¹ and R² represent independently from each other (C₁₋₄)alkyl (especially ethyl); and
R³ represents methyl, ethyl or n-propyl (especially ethyl);
with a hydrolase to give the compound of formula (I) with an enantiomeric excess (ee) of at least 70%.

Definitions provided herein are intended to apply uniformly throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein.

The term “equivalents”, as used in the context of “the amount of a first compound is “X” equivalents relative to the amount of a second compound”, means that a given mixture contains “X” times the amount (in any unity related to the number of molecules) of a first compound relative to the amount of a second compound (given in the same unity).

Unless used regarding temperatures, the term “about” placed before a numerical value “X” refers in the current application to an interval extending from X minus 10% of X to X plus 10% of X, especially to an interval extending from X minus 5% of X to X plus 5% of X and notably to an interval extending from X minus 2% of X to X plus 2% of X. In the particular case of temperatures, the term “about” placed before a temperature “Y” refers in the current application to an interval extending from the temperature Y minus 10° C. to Y plus 10° C., especially to an interval extending from Y minus 5° C. to Y plus 5° C., and notably to an interval extending from Y minus 3° C. to Y plus 3° C. Room temperature means a temperature of about 25° C.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The expression % w/w refers to a percentage by weight compared to the total weight of the composition considered. Likewise, the expression v/v refers to a ratio by volume of one component relative to the total volume (and % v/v refers to the respective ratio in percent).

The term “alkyl”, used alone or in combination, refers to a straight or branched saturated hydrocarbon chain containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, n-propyl, iso-propyl, n-butyl, iso-butyl, sec.-butyl and tert.-butyl. In case “R¹” represents a “(C₁₋₄)alkyl” group, the term “(C₁₋₄)alkyl” means methyl, ethyl, n-propyl, iso-propyl, n-butyl, iso-butyl, sec.-butyl and tert.-butyl, preferably methyl, ethyl, and n-propyl, and most preferably ethyl. In case “R²” represents a “(C₁₋₄)alkyl” group, the term “(C₁₋₄)alkyl” means methyl, ethyl, n-propyl, iso-propyl, n-butyl, iso-butyl, sec.-butyl and tert.-butyl, preferably methyl, ethyl, and n-propyl, and most preferably ethyl.

Any reference to a “hydrolase” that reacts with a compound of formula (II) to give the compound of formula (I) with a given enantiomeric excess, means also that the hydrolase is suitable for providing the compound of formula (I) with that given enantiomeric excess in a reaction with a compound of formula (II).

For the avoidance of doubt, an enantiomeric excess (ee) of the compound of formula (I) of at least 70% means that the ratio between the compound of formula (I) and its enantiomer in a given mixture or product is 85:15 or higher; accordingly, an enantiomeric excess (ee) of the compound of formula (I) of at least 96% means that the ratio between the compound of formula (I) and its enantiomer in a given mixture or product is 98:2 or higher.

It is to be understood that the compound of formula (II) refers to any mixture of the respective (S)-enantiomer and the respective (R)-enantiomer in a ratio between 65:35 and 35:65, notably in a ratio between 55:45 and 45:55, and especially in a ratio between 52:48 and 48:52. Most preferred is a racemic mixture containing the(S)-enantiomer and the (R)-enantiomer in a 1:1 ratio.

The term “salt”, as used in the context of “a compound of formula (I) or of a salt thereof”, refers to the compound of formula (I) wherein the proton of the carboxylic acid function has been replaced by a suitable cation to build a salt of the compound of formula (I). Suitable cations are especially cations with a weight of below 110 g/mol and notably metal cations with a weight of below or equal to 30 g/mol. Preferred are the alkali metal and alkaline earth metal salts of the compound of formula (I); especially the sodium and potassium salts and notably the sodium salt of the compound of formula (I).

2) A further embodiment refers to a process according to embodiment 1), wherein the hydrolase is selected from AH002, AH008, AH012, AH016, AH017, AH018, AH019, AH022, AH023, AH025, AH027, AH028, AH032, AH034, AH035, AH036, AH037, AH041, AH042, AH044, AH045, AH047, AH048, AH051, AH052, AH055, AH056, AH057, AH059, AH060, AH061, AH062, CL055, CL067, Protease M, EU62, DSM-A1, DSM-A2, DSM-A3, DSM-A6, DSM-B1, DSM-B2, DSM-B3, DSM-B6, DSM-C1, DSM-C2, DSM-C3, DSM-D2, and DSM-D3.

AH002, AH008, AH012, AH016, AH017, AH018, AH019, AH022, AH023, AH025, AH027, AH028, AH032, AH034, AH035, AH036, AH037, AH041, AH042, AH044, AH045, AH047, AH048, AH051, AH052, AH055, AH056, AH057, AH059, AH060, AH061, AH062, CL055, and CL067 are commercially available enzymes and may be obtained from Almac; Protease M is commercially available and may be obtained from Amano Enzyme (especially Amano Enzyme Manufacturing, Suqian City, Jiangsu Province, China); EU62 is commercially available and may be obtained from Eucodis Bioscience; DSM-A1, DSM-A2, DSM-A3, DSM-A6, DSM-B1, DSM-B2, DSM-B3, DSM-B6, DSM-C1, DSM-C2, DSM-C3, DSM-D2, and DSM-D3 are commercially available enzymes and may be obtained from DSM Innosyn/Innosyn. All enzymes may be also obtained via Almac.

3) In another embodiment, the present invention relates to a process for the manufacturing of a compound of formula (I), or of a salt thereof,

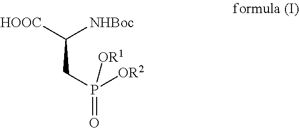

said process comprising the step of reacting a compound of formula (II)

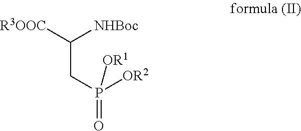

wherein
R¹ and R² represent independently from each other (C₁₋₄)alkyl (especially ethyl); and
R³ represents methyl, ethyl or n-propyl (especially ethyl);
with a hydrolase, wherein the hydrolase is selected from AH002, AH008, AH012, AH016, AH017, AH018, AH019, AH022, AH023, AH025, AH027, AH028, AH032, AH034, AH035, AH036, AH037, AH041, AH042, AH044, AH045, AH047, AH048, AH051, AH052, AH055, AH056, AH057, AH059, AH060, AH061, AH062, CL055, CL067, Protease M, EU62, DSM-A1, DSM-A2, DSM-A3, DSM-A6, DSM-B1, DSM-B2, DSM-B3, DSM-B6, DSM-C1, DSM-C2, DSM-C3, DSM-D2, and DSM-D3.

4) In another embodiment, the present invention relates to a process for the manufacturing of selatogrel

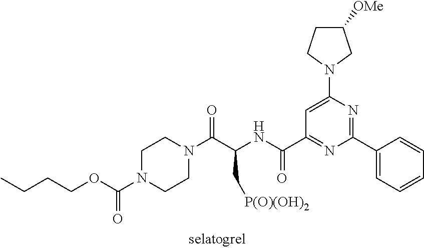

wherein the process comprises the step of reacting a compound of formula (II)

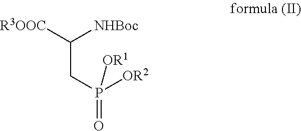

wherein
R¹ and R² represent independently from each other (C₁₋₄)alkyl (especially ethyl); and
R³ represents methyl, ethyl or n-propyl (especially ethyl);
with a hydrolase to give a compound of formula (I), or a salt thereof, with an enantiomeric excess (ee) of at least 70%

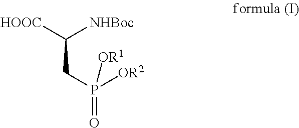

5) A further embodiment refers to a process according to any one of embodiments 1), 3) or 4), wherein the hydrolase is selected from AH012, AH016, AH017, AH018, AH022, AH023, AH025, AH027, AH028, AH032, AH034, AH035, AH036, AH041, AH042, AH044, AH047, AH048, AH051, AH052, AH055, AH059, AH061, and Protease M.

6) A further embodiment refers to a process according to any one of embodiments 1), 3) or 4), wherein the hydrolase is selected from AH017, AH018, AH022, AH023, AH025, AH027, AH032, AH034, AH044, AH047, AH059, AH061, and Protease M.

7) A further embodiment refers to a process according to any one of embodiments 1), 3) or 4), wherein the hydrolase is selected from AH018, AH022, AH023, AH027, AH034, AH044, AH047, and Protease M.

8) A further embodiment refers to a process according to any one of embodiments 1), 3) or 4), wherein the hydrolase is selected from AH018, AH023, AH034, and Protease M.

9) A further embodiment refers to a process according to any one of embodiments 1), 3) or 4), wherein the hydrolase is Protease M.

Protease M (especially Protease M-SD) is commercially available from Amano Enzyme (especially Amano Enzyme Manufacturing, Suqian City, Jiangsu Province, China), is obtained from Aspergillus oryzae through fermentation, and has a high protease and peptidase activity. Protease M-SD refers to spray-dried Protease M.

10) A further embodiment refers to a process according to any one of embodiments 1) to 9), wherein the process gives the compound of formula (I) with an enantiomeric excess (ee) of at least 85%.

11) A further embodiment refers to a process according to any one of embodiments 1) to 9), wherein the process gives the compound of formula (I) with an enantiomeric excess (ee) of at least 96%.

12) A further embodiment refers to a process according to any one of embodiments 1) to 9), wherein the process gives the compound of formula (I) with an enantiomeric excess (ee) of at least 99%.

13) A further embodiment refers to a process according to any one of embodiments 1) to 12), wherein R¹ and R² represent independently from each other methyl, ethyl or n-propyl (especially methyl or ethyl).

14) A further embodiment refers to a process according to any one of embodiments 1) to 12), wherein R¹ and R² represent the same alkyl group selected from methyl and ethyl.

15) A further embodiment refers to a process according to any one of embodiments 1) to 12), wherein R¹ and R² both represent ethyl.

16) A further embodiment refers to a process according to any one of embodiments 1) to 15), wherein R³ represents methyl or ethyl.

17) A further embodiment refers to a process according to any one of embodiments 1) to 15), wherein R³ represents ethyl.

18) A further embodiment refers to a process according to any one of embodiments 1) to 17), wherein the reaction (enzymatic resolution) is conducted in an aqueous solution.

The enzymatic resolution may be conducted in water (especially in purified water) as the only solvent or in a solvent mixture of water and a suitable organic solvent (especially MTBE, DMSO or toluene). The addition of an organic solvent has the beneficial effect of reducing the viscosity of the solution.

19) A further embodiment refers to a process according to any one of embodiments 1) to 17), wherein the reaction (enzymatic resolution) is conducted in a mixture of water and an organic solvent selected from MTBE, DMSO, toluene and any mixture thereof.

20) A further embodiment refers to a process according to any one of embodiments 1) to 17), wherein the reaction (enzymatic resolution) is conducted in a mixture of water and an organic solvent selected from MTBE, DMSO and toluene.

21) A further embodiment refers to a process according to any one of embodiments 1) to 17), wherein the reaction (enzymatic resolution) is conducted in a mixture of water and MTBE.

22) A further embodiment refers to a process according to any one of embodiments 18) to 21), wherein the amount of the organic solvent is between 0% and 20% v/v.

Lower limits of the amount of the organic solvent are 0% v/v, 3% v/v, and 5% v/v, upper limits are 20% v/v, 15% v/v, and 10% v/v. It is to be understood that each lower limit can be combined with each upper limit. Hence all combinations of lower limits and upper limits shall herewith be specifically disclosed. Preferred is an amount of the organic solvent between 5% and 15% v/v.

23) A further embodiment refers to a process according to any one of embodiments 18) to 22), wherein the reaction (enzymatic resolution) is conducted in the presence of a buffer having a pKa value in water at 25° C. between 5.2 and 8.4.

Lower limits of the pKa value of the buffer are 5.2, 5.5, and 6.2, upper limits are 8.4, 7.9 and 7.4. It is to be understood that each lower limit can be combined with each upper limit. Hence all combinations of lower limits and upper limits shall herewith be specifically disclosed. Preferably the buffer is selected from a phosphate buffer and a carbonate buffer. Especially preferred is a phosphate buffer.

24) A further embodiment refers to a process according to any one of embodiments 18) to 23), wherein the reaction (enzymatic resolution) is conducted at a pH value between 5.0 and 8.0.

Lower limits of the pH value are 5.0, 6.0, 6.5, and 6.7, upper limits are 8.0, 7.4, 7.0 and 6.9. It is to be understood that each lower limit can be combined with each upper limit. Hence all combinations of lower limits and upper limits shall herewith be specifically disclosed. Preferably the pH value is between 6.5 and 7.0 and especially between 6.7 and 6.9.

25) A further embodiment refers to a process according to any one of embodiments 18) to 24), wherein the pH value of the solution is kept in a range of plus/minus 0.2 (preferably 0.1) during the reaction (enzymatic resolution) by addition of a base.

The base may be added sequentially or continuously to the reaction mixture. Preferably the base is added as an aqueous solution, especially as an aqueous solution of potassium carbonate. It is preferred that the pH value of the solution is kept in a range between 6.6 and 7.0 (most preferably between 6.7 and 6.9).

26) A further embodiment refers to a process according to any one of embodiments 1) to 25), wherein the reaction (enzymatic resolution) is conducted at a temperature between 10° C. and 60° C.

Lower limits of the reaction temperature are 10° C., 15° C., 20° C., and 25° C., upper limits are 60° C., 40° C., 33° C., and 30° C. It is to be understood that each lower limit can be combined with each upper limit. Hence all combinations of lower limits and upper limits shall herewith be specifically disclosed. Preferably the reaction temperature is between 20° C. and 33° C. and especially between 25° C. and 30° C.

27) A further embodiment refers to a process according to any one of embodiments 1) to 26), wherein the reaction (enzymatic resolution) is conducted with an enzyme loading of between 0.05% and 100%.

The term “enzyme loading” refers to the ratio by weight of the amount of enzyme and the used amount of substrate, i.e. the used amount of compound of formula (II). For instance, an enzyme loading of 1% means that 10 mg enzyme are used in the reaction per 1 g of compound of formula (II). Lower limits of the enzyme loading are 0.05%, 0.2%, 0.5%, and 0.8%, upper limits are 100%, 20%, 10%, and 2%. It is to be understood that each lower limit can be combined with each upper limit. Hence all combinations of lower limits and upper limits shall herewith be specifically disclosed. Preferably the enzyme loading is between 0.2% and 10% and especially between 0.5% and 2.0%.

28) A further embodiment refers to a process according to any one of embodiments 1) to 27), wherein the reaction (enzymatic resolution) is conducted with a substrate loading between 5.0 g substrate per liter (L) water and 300 g substrate per liter (L) water in the reaction mixture. The term “substrate loading” refers to the used gram-amount of substrate, i.e. the used gram-amount of compound of formula (II), per volume water in liters in the reaction mixture. For the avoidance of doubt, only the volume of water at the beginning of the reaction is considered, but not the volume of water that is added together with a base to keep the pH value constant during the course of the reaction. Lower limits of the substrate loading are 5.0 g substrate per L water, 20 g substrate per L water, and 100 g substrate per L water, upper limits are 300 g substrate per L water, 220 g substrate per L water, and 150 g substrate per L water. It is to be understood that each lower limit can be combined with each upper limit. Hence all combinations of lower limits and upper limits shall herewith be specifically disclosed. Preferably the substrate loading is between 20 g substrate per L water and 220 g substrate per L water.

29) A further embodiment refers to a process according to any one of embodiments 1) to 28), wherein a salt of a divalent metal cation (especially a chloride salt of a divalent metal cation) is added to the reaction mixture.

Preferred salts of a divalent metal cation are ZnCl₂, FeCl₂, MnSO₄, CoCl₂, MgCl₂, CaCl₂), and NiCl₂ (especially ZnCl₂, FeCl₂, and MnSO₄). The salts may be used in a concentration between 0.5 mM and 5.0 mM (especially between 1.0 mM and 3.0 mM).

30) A further embodiment refers to a process according to any one of embodiments 1) to 29), wherein the reaction time of the reaction (enzymatic resolution) is between 5 hours and 36 hours.

The term “reaction time” refers to the time between addition of the last reagent to the reaction mixture until start of the work-up procedure, for instance by extracting the reaction mixture with an organic solvent. Lower limits of the reaction time are 5 hours, 10 hours, and 18 hours, upper limits are 36 hours, 28 hours, and 24 hours. It is to be understood that each lower limit can be combined with each upper limit. Hence all combinations of lower limits and upper limits shall herewith be specifically disclosed.

31) A further embodiment refers to a process according to embodiment 30), wherein the reaction time of the reaction (enzymatic resolution) is between 10 hours and 28 hours.

32) A further embodiment refers to a process according to any one of embodiments 1) to 31), wherein the process comprises a work-up of the reaction mixture by a process comprising the steps of addition of an aqueous solution of a base (especially an aqueous potassium carbonate solution) to adjust the pH value to between 7.4 and 9.0 (especially between 7.5 and 8.5 and notably between 7.6 and 8.0); addition of an organic solvent (especially MTBE); separation of the organic and the aqueous layer; addition of an aqueous solution of an acid (especially hydrochloric acid) to the aqueous layer to adjust the pH value to between 1.0 and 4.0 (especially between 1.2 and 2.0 and notably between 1.4 and 1.6); and isolation of the precipitated compound of formula (I).

33) A further embodiment refers to a process according to any one of embodiments 1) to 32), wherein the process comprises the further step of recrystallizing the compound of formula (I) from 2-propanol.

(R)-2-((tert-butoxycarbonyl)amino)-3-(diethoxyphosphoryl)-propanoic acid may be recrystallized from solvent selected from methanol, ethanol, 2-propanol or any mixture thereof. The recrystallization solution may contain up to 20% w/w water. Preferably the recrystallization solution contains less than 5% w/w water. As the presence of water results in lower yields, it is preferred that the water content in the recrystallization is as low as possible. The recrystallization has the advantage to reduce the protein (enzyme) content. The phrase “solvent selected from methanol, ethanol, 2-propanol or any mixture thereof”, means that the solvent is methanol, ethanol, 2-propanol, or a mixture of two or three of methanol, ethanol, and 2-propanol. In case the solvent is a mixture, it is preferred that the solvent is a mixture of two of methanol, ethanol, and 2-propanol.

34) A further embodiment refers to a process according to any one of embodiments 1) to 33), wherein the process gives (R)-2-((tert-butoxycarbonyl)amino)-3-(diethoxyphosphoryl)-propanoic acid in crystalline form (I).

35) A further embodiment refers to a process according to embodiment 34), wherein (R)-2-((tert-butoxycarbonyl)amino)-3-(diethoxyphosphoryl)propanoic acid in crystalline form (I) is characterized by the presence of peaks in the X-ray powder diffraction diagram at the following angles of refraction 2θ: 9.8°, 10.3°, and 17.5°.

36) A further embodiment refers to a process according to embodiment 34), wherein (R)-2-((tert-butoxycarbonyl)amino)-3-(diethoxyphosphoryl)propanoic acid in crystalline form (I) is characterized by the presence of peaks in the X-ray powder diffraction diagram at the following angles of refraction 2θ: 9.8°, 10.3°, 16.2°, 17.5°, and 27.3°.

37) A further embodiment refers to a process according to embodiment 34), wherein (R)-2-((tert-butoxycarbonyl)amino)-3-(diethoxyphosphoryl)propanoic acid in crystalline form (I) is characterized by the presence of peaks in the X-ray powder diffraction diagram at the following angles of refraction 2θ: 9.8°, 10.3°, 12.5°, 16.2°, 17.5°, 20.7°, 22.4°, 24.2°, 24.7°, and 27.3°.

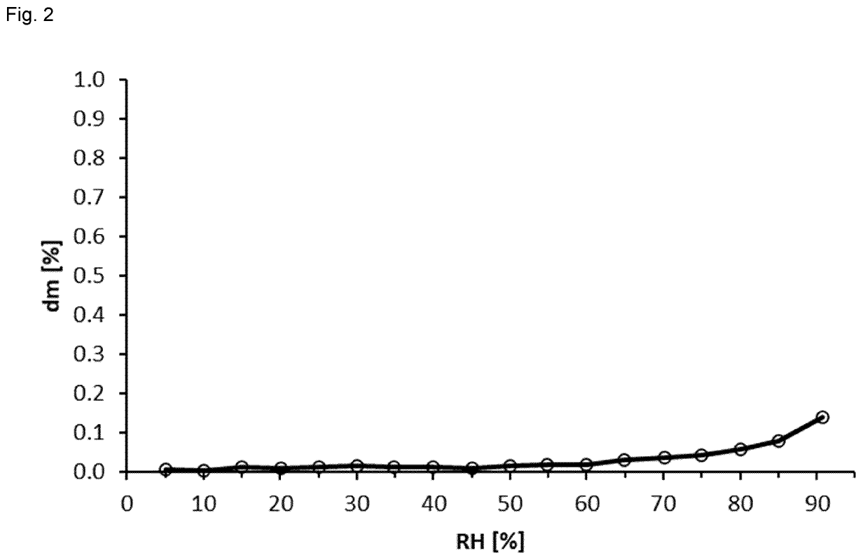

38) A further embodiment refers to a process according to any one of embodiments 34) to 37), wherein (R)-2-((tert-butoxycarbonyl)amino)-3-(diethoxyphosphoryl)propanoic acid in crystalline form (I) is characterized by essentially showing a gravimetric moisture sorption profile (sorption cycle) as depicted in FIG. 2, wherein the gravimetric moisture sorption profile is measured at 25° C.

39) A further embodiment refers to a process according to any one of embodiments 1) to 38), wherein the process further comprises the step of reacting a compound of formula (III)

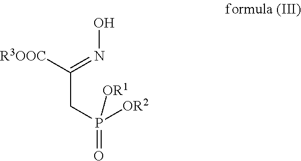

with hydrogen in the presence of palladium on charcoal and Boc₂O to give a compound of formula (II)

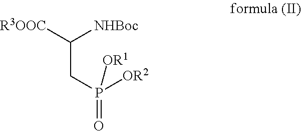

wherein
R¹ and R² represent independently from each other (C₁₋₄)alkyl (especially ethyl); and
R³ represents methyl, ethyl or n-propyl (especially ethyl);

40) A further embodiment refers to a process according to embodiment 39), wherein R¹, R² and R³ represent ethyl.

41) A further embodiment refers to a process according to any one of embodiments 39) or 40), wherein the process is conducted in a solvent selected from methanol, ethanol and 2-propanol (especially ethanol).

42) A further embodiment refers to a process according to any one of embodiments 39) to 41), wherein the hydrogenation is conducted at a temperature between 50° C. and 70° C. (especially between 55° C. and 65° C.).

43) A further embodiment refers to a process according to any one of embodiments 1) to 42), wherein the process further comprises the steps of reacting a compound of formula (IV)

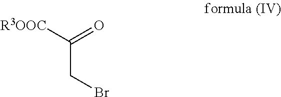

sequentially with hydroxylamine hydrochloride and trialkylphosphite (especially triethylphosphite) to give a compound of formula (III)

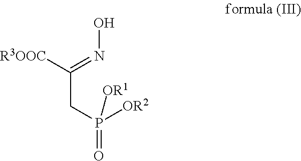

wherein
R¹ and R² represent independently from each other (C₁₋₄)alkyl (especially ethyl); and
R³ represents methyl, ethyl or n-propyl (especially ethyl);

44) A further embodiment refers to a process according to embodiment 43), wherein R¹, R² and R³ represent ethyl.

45) A further embodiment refers to a process according to any one of embodiments 1) to 44), wherein the process further comprises the steps of reacting a compound of formula (V)

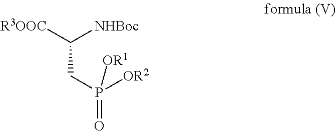

with a base (especially NaOEt) to give a compound of formula (II)

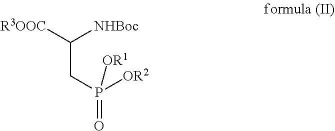

wherein
R¹ and R² represent independently from each other (C₁₋₄)alkyl (especially ethyl); and
R³ represents methyl, ethyl or n-propyl (especially ethyl);
and wherein the compound of formula (V) has an enantiomeric excess (ee) of at least 80% and the compound of formula (II) has an enantiomeric excess (ee) of less than 10% (and especially less or equal than 2%).

The enzymatic resolution of a compound of formula (II) that is disclosed in this specification, and especially in embodiments 1) to 31), results (before a work-up of the crude reaction mixture) in a mixture of a compound of formula (I) (especially (R)-2-((tert-butoxycarbonyl)amino)-3-(diethoxyphosphoryl)propanoic acid and a compound of formula (V) (especially ethyl(S)-2-((tert-butoxycarbonyl)amino)-3-(diethoxy-phosphoryl)propanoate). The crude mixture of the acid and the ester may be separated by any suitable method like extraction; precipitation of one component or chromatography. Especially, the crude mixture of the acid and the ester may be separated by partition between an aqueous layer having a pH value higher than 7.4 (containing the acid) and an organic layer (containing the ester). Preferred organic solvents for the partition/extraction are ether and especially MTBE. The ester (especially ethyl(S)-2-((tert-butoxycarbonyl)amino)-3-(diethoxy-phosphoryl)-propanoate) may be isolated by removal of the organic solvent from the extraction and may be used in a recycling procedure with a base to give racemic compound of formula (II) (especially ethyl 2-((tert-butoxycarbonyl)amino)-3-(diethoxy-phosphoryl)propanoate) that may be used again in the enzymatic resolution.

46) A further embodiment refers to a process according to embodiment 45), wherein the enantiomeric excess (ee) of the compound of formula (V) is at least 90% (and especially at least 96%).

47) A further embodiment refers to a process according to any one of embodiments 45) or 46), wherein the process gives the compound of formula (II) with an enantiomeric excess (ee) of less than 4% (and especially less or equal than 2%).

48) A further embodiment refers to a process according to any one of embodiments 45) to 47), wherein R¹ and R² represent independently from each other methyl, ethyl or n-propyl (especially methyl or ethyl).

49) A further embodiment refers to a process according to any one of embodiments 45) to 47), wherein R¹ and R² represent the same alkyl group selected from methyl and ethyl.

50) A further embodiment refers to a process according to any one of embodiments 45) to 47), wherein R¹ and R² both represent ethyl.

51) A further embodiment refers to a process according to any one of embodiments 45) to 50), wherein R³ represents methyl or ethyl.

52) A further embodiment refers to a process according to any one of embodiments 45) to 50), wherein R³ represents ethyl.

53) A further embodiment refers to a process according to any one of embodiments 45) to 52), wherein the reaction (racemization) is conducted in a solvent selected from diethylether, MTBE, THF or 2-methyl-tetrahydrofuran.

54) A further embodiment refers to a process according to any one of embodiments 45) to 52), wherein the reaction (racemization) is conducted in MTBE.

55) A further embodiment refers to a process according to any one of embodiments 45) to 54), wherein the base is selected from sodium ethoxide (NaOEt) and potassium tert-butoxide (KOtBu) (and especially sodium ethoxide).

56) A further embodiment refers to a process according to any one of embodiments 45) to 54), wherein the base is a solution of sodium ethoxide (NaOEt) in ethanol.

57) A further embodiment refers to a process according to any one of embodiments 55) to 56), wherein the amount of base is between 0.1 eq and 1.0 eq (especially between 0.4 eq and 0.8 eq, and notably between 0.5 eq and 0.7 eq) relative to the amount of compound of formula (V).

58) A further embodiment refers to a process according to any one of embodiments 45) to 57), wherein the reaction (racemization) is conducted at a reaction temperature between 0° C. and 30° C. (especially between 0° C. and 10° C. and notably between 0° C. and 5° C.).

59) A further embodiment refers to a process according to any one of embodiments 45) to 58), wherein the water content in the reaction mixture of the reaction (racemization) is ≤0.5% v/v (especially ≤0.2% v/v, and notably ≤0.1% v/v)

60) Another embodiment of the invention relates to a crystalline form of (R)-2-((tert-butoxycarbonyl)amino)-3-(diethoxyphosphoryl)propanoic acid (crystalline form (I)), characterized by the presence of peaks in the X-ray powder diffraction diagram at the following angles of refraction 2θ: 9.8°, 10.3°, and 17.5°.

It is understood that the crystalline form according to embodiment 60) comprise COMPOUND in free form (non-salt form). Furthermore, said crystalline form may comprise non-coordinated and/or coordinated solvent (especially non-coordinated and/or coordinated water).

Coordinated solvent (especially coordinated water) is used herein as term for a crystalline solvate (especially a crystalline hydrate). For the avoidance of doubt, in this application the term “crystalline hydrate” encompasses stoichiometric and non-stoichiometric hydrates (especially stoichiometric hydrates). Likewise, non-coordinated solvent is used herein as term for physiosorbed or physically entrapped solvent (definitions according to Polymorphism in the Pharmaceutical Industry (Ed. R. Hilfiker, VCH, 2006), Chapter 8: U. J. Griesser: The Importance of Solvates).

61) Another embodiment of the invention relates to a crystalline form of (R)-2-((tert-butoxycarbonyl)amino)-3-(diethoxyphosphoryl)propanoic acid (crystalline form (I)) according to embodiment 60), characterized by the presence of peaks in the X-ray powder diffraction diagram at the following angles of refraction 2θ: 9.8°, 10.3°, 16.2°, 17.5°, and 27.3°.

62) Another embodiment of the invention relates to a crystalline form of (R)-2-((tert-butoxycarbonyl)amino)-3-(diethoxyphosphoryl)propanoic acid (crystalline form (I)) according to embodiment 60), characterized by the presence of peaks in the X-ray powder diffraction diagram at the following angles of refraction 2θ: 9.8°, 10.3°, 12.5°, 16.2°, 17.5°, 20.7°, 22.4°, 24.2°, 24.7°, and 27.3°.

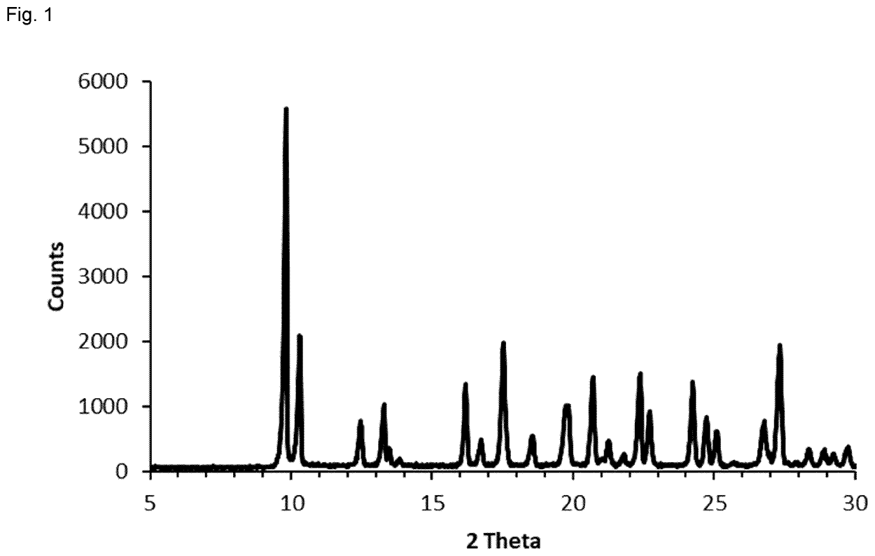

63) Another embodiment of the invention relates to a crystalline form of (R)-2-((tert-butoxycarbonyl)amino)-3-(diethoxyphosphoryl)propanoic acid (crystalline form (I)) according to any one of embodiments 60) to 62), which essentially shows the X-ray powder diffraction pattern as depicted in FIG. 1.

64) Another embodiment of the invention relates to a crystalline form of (R)-2-((tert-butoxycarbonyl)amino)-3-(diethoxyphosphoryl)propanoic acid (crystalline form (I)) according to any one of embodiments 60) to 63), characterized by an endothermic peak at about 180° C. as measured by DSC (notably at 180° C.±1° C., and especially at 180° C.).

65) Another embodiment of the invention relates to a crystalline form of (R)-2-((tert-butoxycarbonyl)amino)-3-(diethoxyphosphoryl)propanoic acid (crystalline form (I)) according to any one of embodiments 60) to 64), which essentially shows a gravimetric moisture sorption profile as depicted in FIG. 2, wherein the gravimetric moisture sorption profile is measured at 25° C.

66) Another embodiment of the invention relates to a crystalline form of ethyl 3-(diethoxyphosphoryl)-2-(hydroxyimino)propanoate (crystalline form (A)), characterized by the presence of peaks in the X-ray powder diffraction diagram at the following angles of refraction 2θ: 10.2°, 11.3°, and 22.7°.

It is understood that the crystalline form according to embodiment 66) comprise ethyl 3-(diethoxyphosphoryl)-2-(hydroxyimino)propanoate in free form (non-salt form). Furthermore, said crystalline form may comprise non-coordinated and/or coordinated solvent (especially non-coordinated and/or coordinated water). Coordinated solvent (especially coordinated water) is used herein as term for a crystalline solvate (especially a crystalline hydrate). For the avoidance of doubt, in this application the term “crystalline hydrate” encompasses stoichiometric and non-stoichiometric hydrates (especially stoichiometric hydrates). Likewise, non-coordinated solvent is used herein as term for physiosorbed or physically entrapped solvent (definitions according to Polymorphism in the Pharmaceutical Industry (Ed. R. Hilfiker, VCH, 2006), Chapter 8: U. J. Griesser: The Importance of Solvates).

67) Another embodiment of the invention relates to a crystalline form of ethyl 3-(diethoxyphosphoryl)-2-(hydroxyimino)propanoate (crystalline form (A)) according to embodiment 66), characterized by the presence of peaks in the X-ray powder diffraction diagram at the following angles of refraction 2θ: 10.2°, 11.3°, 13.2°, 22.7°, and 22.9°.

68) Another embodiment of the invention relates to a crystalline form of ethyl 3-(diethoxyphosphoryl)-2-(hydroxyimino)propanoate (crystalline form (A)) according to embodiment 66), characterized by the presence of peaks in the X-ray powder diffraction diagram at the following angles of refraction 2θ: 10.2°, 11.3°, 13.2°, 13.8°, 15.2°, 20.5°, 22.7°, 22.9°, 25.3°, and 26.6°.

69) Another embodiment of the invention relates to a crystalline form of ethyl 3-(diethoxyphosphoryl)-2-(hydroxyimino)propanoate (crystalline form (A)) according to any one of embodiments 66) to 68), which essentially shows the X-ray powder diffraction pattern as depicted in FIG. 4.

70) Another embodiment of the invention relates to a crystalline form of ethyl 3-(diethoxyphosphoryl)-2-(hydroxyimino)propanoate (crystalline form (B)), characterized by the presence of peaks in the X-ray powder diffraction diagram at the following angles of refraction 2θ: 10.9°, 19.5°, and 27.2°.

It is understood that the crystalline form according to embodiment 70) comprise ethyl 3-(diethoxyphosphoryl)-2-(hydroxyimino)propanoate in free form (non-salt form). Furthermore, said crystalline form may comprise non-coordinated and/or coordinated solvent (especially non-coordinated and/or coordinated water). Coordinated solvent (especially coordinated water) is used herein as term for a crystalline solvate (especially a crystalline hydrate). For the avoidance of doubt, in this application the term “crystalline hydrate” encompasses stoichiometric and non-stoichiometric hydrates (especially stoichiometric hydrates). Likewise, non-coordinated solvent is used herein as term for physiosorbed or physically entrapped solvent (definitions according to Polymorphism in the Pharmaceutical Industry (Ed. R. Hilfiker, VCH, 2006), Chapter 8: U. J. Griesser: The Importance of Solvates).

71) Another embodiment of the invention relates to a crystalline form of ethyl 3-(diethoxyphosphoryl)-2-(hydroxyimino)propanoate (crystalline form (B)) according to embodiment 70), characterized by the presence of peaks in the X-ray powder diffraction diagram at the following angles of refraction 2θ: 10.9°, 18.2°, 19.5°, 27.2°, and 29.5°.

72) Another embodiment of the invention relates to a crystalline form of ethyl 3-(diethoxyphosphoryl)-2-(hydroxyimino)propanoate (crystalline form (B)) according to embodiment 70), characterized by the presence of peaks in the X-ray powder diffraction diagram at the following angles of refraction 2θ: 10.9°, 18.2°, 19.5°, 19.8°, 25.3°, 27.2°, 29.5°, 33.0°, 34.7°, and 36.9°.

73) Another embodiment of the invention relates to a crystalline form of ethyl 3-(diethoxyphosphoryl)-2-(hydroxyimino)propanoate (crystalline form (B)) according to any one of embodiments 70) to 72), which essentially shows the X-ray powder diffraction pattern as depicted in FIG. 5.

For avoidance of any doubt, whenever one of the above embodiments refers to “peaks in the X-ray powder diffraction diagram at the following angles of refraction 20”, said X-ray powder diffraction diagram is obtained by using combined Cu Kα1 and Kα2 radiation, without Kα2 stripping; and it should be understood that the accuracy of the 20 values as provided herein is in the range of +/−0.1-0.2°. Notably, when specifying an angle of refraction 2 theta (2θ) for a peak in the invention embodiments and the claims, the 20 value given is to be understood as an interval from said value minus 0.2° to said value plus 0.2° (20+/−) 0.2°; and preferably from said value minus 0.1° to said value plus 0.1° (20+/−0.1°).

When defining the presence of peak in e.g. an X-ray powder diffraction diagram, a common approach is to do this in terms of the S/N ratio (S=signal, N=noise). According to this definition, when stating that a peak has to be present in an X-ray powder diffraction diagram, it is understood that the peak in the X-ray powder diffraction diagram is defined by having an S/N ratio (S=signal, N=noise) of greater than x (x being a numerical value greater than 1), usually greater than 2, especially greater than 3.

In the context with stating that the crystalline form essentially shows an X-ray powder diffraction pattern as depicted in FIG. 1, 4, or 5, respectively, the term “essentially” means that at least the major peaks of the diagram depicted in said figures, i.e. those having a relative intensity of more than 20%, especially more than 10%, as compared to the most intense peak in the diagram, have to be present. However, the person skilled in the art of X-ray powder diffraction will recognize that relative intensities in X-ray powder diffraction diagrams may be subject to strong intensity variations due to preferred orientation effects.

### Abbreviations and Terms Used in this Text

### Abbreviations

The following abbreviations are used throughout the specification and the examples:
Ac acetyl
AcCN acetonitrile
AcOH acetic acid
(L-) Ala (L-) alanine
aq aqueous
Boc tert.-butyloxycarbonyl
DSC differential scanning calorimetry
ee enantiomeric excess
eq. equivalent(s)
Et ethyl
EtOH ethanol
GVS gravimetric vapour sorption
h hour(s)
HPLC high performance liquid chromatography
IPA 2-propanol
JT jacket temperature
M molarity/molar concentration
Me methyl
min minute(s)
MTBE methyl tert-butyl ether
NaOEt sodium ethoxide
PDA photodiode-array detector
RBF round bottom flask
rpm revolutions per minute
rt retention time
RT room temperature
THF tetrahydrofuran
TFA trifluoroacetic acid
vol 1 vol means 1 L solvent per 1 kg reference starting material
% a/a percent determined by area ratio

### Experimental Part

### X-Ray Powder Diffraction Analysis (XRPD)

X-ray powder diffraction patterns were collected on a Bruker D8 Advance X-ray diffractometer equipped with a Lynxeye detector operated in reflection mode (coupled two Theta/Theta). Typically, the Cu X-ray tube was run at of 40 kV/40 mA. A step size of 0.02° (2θ) and a step time of 0.04 sec per step over a scanning range of 3-50° in 2θ were applied. The divergence slit was set to fixed sample illumination (variable slit size) and the antiscatter slit was set to 0.3°. Powders were slightly pressed into a silicon single crystal sample holder with depth of 0.5 mm and samples were rotated in their own plane during the measurement. Diffraction data are reported using Cu Kα (λ=1.5418 Å) radiation. The accuracy of the 20 values as provided herein is in the range of +/−0.1-0.2° as it is generally the case for conventionally recorded X-ray powder diffraction patterns.

### Differential Scanning Calorimetry (DSC)

DSC data were collected on a Mettler Toledo STARe System (DSC3 module, measuring cell with ceramic sensor and STAR software version 16.00b) equipped with a 34 position auto-sampler. The instrument was calibrated for energy and temperature using certified indium. Typically, 1-5 mg of each sample, in an automatically pierced aluminium pan, was heated at 10° C. min⁻¹, unless stated otherwise, from −20° C. to 250° C. A nitrogen purge at 20 mL min⁻¹ was maintained over the sample. Peak temperatures are reported for melting points.

### Gravimetric Vapour Sorption (GVS) Analysis

Measurements were performed on a multi sample instrument SPS-100n (ProUmid GmbH, Ulm, Germany) operated in stepping mode at 25° C. The sample was allowed to equilibrate at 40% RH before starting a pre-defined humidity program (40-0-95-40% RH, steps of 5% ΔRH and with a maximal equilibration time of 24 hours per step were applied. About 20 to 30 mg of each sample was used. The hygroscopic classification is done according to the European Pharmacopeia Technical Guide (1999, page 86), e.g., not hygroscopic: increase in mass is less than 0.2% mass/mass; slightly hygroscopic: increase in mass is less than 2% and equal to or greater than 0.2% mass/mass; hygroscopic: increase in mass is less than 15% and equal to or greater than 2% mass/mass. The mass change between 40% relative humidity and 80% relative humidity in the first adsorption scan is considered.

### High Performance Liquid Chromatography (HPLC):

### HPLC (Method 1):

HPLC system: Agilent 1260 series system
Flow: 1.0 mL/min
Column temperature: 30° C.
Autosampler temperature: RT
Injection volume: 5 μL
Column: Zorbax SB-Aq, 150×4.6 mm, 3.5 μm
Detection wavelength (UV): 210 nm
Solvent A: 10 mM aq. K₂HPO₄ solution (adjusted to pH 7.0 with 85% phosphoric acid
Solvent B: AcCN
Gradient:

| Time (min) | % solvent A | % solvent B |
|---|---|---|
| 0.0 | 90 | 10 |
| 20.0 | 30 | 70 |
| 20.1 | 90 | 10 |
| 25.0 | 90 | 10 |

### HPLC (Method 2):

HPLC system: Agilent 1260 series system
Flow: 1.0 mL/min
Column temperature: 30° C.
Autosampler temperature: RT
Injection volume: 5 μL
Column: Zorbax SB-Aq, 150×4.6 mm, 3.5 μm
Detection wavelength (UV): 210 nm
Solvent A: 10 mM aq. K₂HPO₄ solution (adjusted to pH 7.0 with 85% phosphoric acid
Solvent B: AcCN
Gradient:

| Time (min) | % solvent A | % solvent B |
|---|---|---|
| 0.0 | 90 | 10 |
| 20.0 | 30 | 70 |
| 25.0 | 30 | 70 |
| 25.1 | 90 | 10 |
| 33.0 | 90 | 10 |

### HPLC (Method 3):

HPLC system: Agilent 1260 series system
Flow: 1.0 mL/min
Column temperature: 40° C.
Autosampler temperature: RT
Injection volume: 5 μL
Column: Atlantis T3, 150×4.6 mm, 3 μm
Detection wavelength (UV): 215 nm
Solvent A: aqueous solution of H₃PO₄ (1% v/v)
Solvent B: AcCN
Gradient:

| Time (min) | % solvent A | % solvent B |
|---|---|---|
| 0.0 | 95 | 5 |
| 25.0 | 50 | 50 |
| 30.0 | 10 | 90 |
| 32.0 | 10 | 90 |
| 32.1 | 95 | 5 |
| 40.0 | 95 | 5 |

### Chiral HPLC:

### Chiral HPLC (Method 1):

HPLC system: Agilent 1260 series system
Flow: 0.9 mL/min
Column temperature: 27° C.
Autosampler temperature: RT
Injection volume: 20 HL
Column: Chiralpak IC-3, 250×4.6 mm, 3 μm
Detection wavelength (UV): 215 nm
Run time 45 min
Mobile phase 0.05% TFA-AcCN/0.05% TFA-H₂O=30/70

### Chiral HPLC (Method 2):

Flow: 1 mL/min
Column temperature: 35° C.
Sample temperature: 35° C.
Injection volume: 30 μL
Column: Chiralpak IC, 250×4.6 mm, 5μ m
Detection wavelength (UV): 220 nm
Run time 40 min
Mobile phase 20% EtOH in heptane (+0.1% TFA)

### Chiral HPLC (Method 3):

Flow: 1 mL/min
Column temperature: 25° C.
Sample temperature: 25° C.
Injection volume: 30 μL
Column: Chiralpak IC-3, 250×4.6 mm, 5 μm
Detection wavelength (PDA): 215 nm
Run time 32 min
Mobile phase 70:30 water (0.05% TFA): CH₃CN (0.05% TFA)

### Example 1

### Synthesis of ethyl 3-bromo-2-(hydroxyimino)propanoate

Hydroxylamine hydrochloride (81.4 kg) and water (460 kg) were charged into a reactor, and seeds of ethyl 3-bromo-2-(hydroxyimino)propanoate (69.0 g) were added. The mixture was stirred at 20 to 25° C. for 0.5 h and ethyl 3-bromo-2-oxopropanoate (230 kg, 1.0 eq.) was added over 1 h at 20 to 25° C. The reaction mixture was stirred for 2 to 3 h, toluene (905 kg) was added and the mixture was stirred at 20 to 25° C. for 4 to 5 h. The layers were separated, water (368 kg) was added to the organic layer, the mixture was stirred for 0.5 h at 20 to 25° C., and the layers were separated. The organic layer was concentrated to 1.0-2.0 vol. at 45 to 50° C. (jacket temperature) and warmed to 45 to 50° C. (internal temperature). Seeds of ethyl 3-bromo-2-(hydroxyimino)propanoate (138 g) were added, the mixture was stirred for 0.5 h at 45 to 50° C., and n-heptane (840 kg) was added to the mixture at 45 to 50° C. within 2 to 4 h. The mixture was cooled to 0 to 5° C. within 6 to 8 h, stirred at 0 to 5° C. for 4 to 5 h, and centrifuged. The solid was washed with n-heptane (88.4 kg) and dried at 30 to 35° C. for 24 h under vacuum to give the product (136 kg).

### Example 2

### Synthesis of ethyl 3-(diethoxyphosphoryl)-2-(hydroxyimino)propanoate

Triethylphosphite (129 kg, 1.2 eq.) and n-heptane (223 kg) were charged in a reactor and the internal temperature was adjusted to 70 to 75° C. Ethyl 3-bromo-2-(hydroxyimino)propanoate (136 kg, 1.0 eq.) was dissolved in isopropyl acetate (285 kg) at 20 to 25° C., and the solution was slowly added to the reaction at 70 to 75° C. in 2 to 4 h. The reaction mixture was stirred at 70 to 75° C. for 10 to 12 h and cooled to 20 to 25° C. Seeds of ethyl 3-(diethoxyphosphoryl)-2-(hydroxyimino)propanoate (81.3 g) were added to the mixture at 20 to 25° C., the mixture was stirred for 1 to 2 h, and n-heptane (461 kg) was added at 20 to 25° C. in 1 to 2 h. The mixture was cooled to 0 to 5° C., stirred for 2 to 3 h, and centrifuged. The obtained solid was washed with n-heptane (184 kg) and dried in vacuo at 20 to 25° C. (JT) for 24 h to give the product (150 kg). Depending on the exact amounts of the solvents, the temperature profile during the crystallization process and the seeding, the product is obtained in either crystalline form (A) or crystalline form (B).

TABLE 1
Characterisation data for ethyl 3-(diethoxyphosphoryl)-
2-(hydroxyimino)-propanoate in crystalline form (A)

| Technique | Data Summary | Remarks |
|---|---|---|
| XRPD | Crystalline | see FIG. 4 |
| | | and FIG. 5 |
| Purity, | 99.6% | |
| HPLC (method 1) | rt = 8.3 min | |

### Example 3

### Synthesis of ethyl 2-((tert-butoxycarbonyl)amino)-3-(diethoxyphosphoryl)propanoate

Ethyl 3-(diethoxyphosphoryl)-2-(hydroxyimino)propanoate (105 kg, 1.0 eq.), EtOH (158 kg) and 90% Boc₂O in THF (116 kg, 1.2 eq) were charged in a 2000 L autoclave. A mixture of 20% Pd/C (2.62 kg) and EtOH (131 kg) was prepared in a container and added into the autoclave. The container was washed with EtOH (105 kg) and the EtOH was added to the autoclave. The pressure in the autoclave was reduced to 50 to 100 mbar, and increased to 0.2 MPa with nitrogen, and the process was repeated three times. The pressure in the autoclave was reduced to 50 to 100 mbar. Then the pressure in the autoclave was increased to 0.2 MPa with hydrogen, reduced to 0.02 MPa, and the process was repeated three times. The mixture was warmed to 55-65° C. with pressurizing the autoclave to 0.4 to 0.5 MPa within 0.5 to 1.0 h. The autoclave was pressurized to 1.4 to 1.5 MPa and the mixture was stirred at 55 to 65° C. for 1.0 h. The hydrogen supply was stopped, and the mixture was cooled to 20 to 25° C. The pressure in the autoclave was reduced to 0.02 MPa. Then the pressure in the autoclave was increased to 0.2 MPa with nitrogen, reduced to 0.02 MPa, and the process was repeated three times. The pressure in the autoclave was increased to 0.2 MPa with hydrogen, reduced to 0.02 MPa, and the process was repeated three times. The mixture was warmed to 55-65° C. with pressurizing the autoclave to 0.4 to 0.5 MPa within 0.5 to 1.0 h. The autoclave was pressurized to 1.4 to 1.5 MPa and the mixture was stirred at 55 to 65° C. for 6.0 h. The hydrogen supply was stopped, and the mixture was cooled to 20 to 25° C. The pressure in the autoclave was reduced to 0.02 MPa. Then the pressure in the autoclave was increased to 0.2 MPa with nitrogen, reduced to 0.02 MPa, and the process was repeated three times. The pressure in the autoclave was increased to 0.2 MPa with hydrogen, reduced to 0.02 MPa, and the process was repeated three times. The mixture was warmed to 55-65° C. with pressurizing the autoclave to 0.4 to 0.5 MPa within 0.5 to 1.0 h. The autoclave was pressurized to 1.4 to 1.5 MPa and the mixture was stirred at 55 to 65° C. for 4.0 h. The hydrogen supply was stopped, and the mixture was cooled to 20 to 25° C. The pressure in the autoclave was reduced to 0.02 MPa, increased to 0.2 MPa with nitrogen, and the process was repeated three times. The pressure was released, and the mixture was filtered through celite. The cake was washed with EtOH (105 kg) and the solution was concentrated to dryness at 45 to 55° C. (jacket temperature). MTBE (210 kg) was added to the residue, and the mixture was concentrated to dryness at 45-55° C. (jacket temperature) and the process was repeated twice. The residue was dissolved in MTBE (68.3 kg), and the solution was stirred for 0.5 h at 20 to 30° C. The solution of ethyl 2-((tert-butoxycarbonyl)amino)-3-(diethoxyphosphoryl)propanoate in MTBE was used for next step.

HPLC (method 2): rt=13.6 min

### Example 4

### Synthesis of (R)-2-((tert-butoxycarbonyl)amino)-3-(diethoxyphosphoryl)propanoic acid

In a 3000 L reactor, KH₂PO₄ (19.4 kg) was dissolved in water (1403 kg) at 25 to 30° C. An aqueous K₂CO₃ solution (20%) was added at 25 to 30° C. to the mixture to adjust the pH value to 6.7 to 6.9. In a separate vessel, ethyl 2-((tert-butoxycarbonyl)amino)-3-(diethoxy-phosphoryl)propanoate (281 kg) was dissolved in MTBE (135 kg) at 25 to 30° C. and added to the reactor. The vessel was washed with MTBE (30.9 kg) and the wash liquid was added to the reaction mixture. Protease M-SD (3.1 kg, Protease M in spray-dried form, Amano) was dissolved in water (31.0 kg) and added to the mixture at 25 to 30° C. The reaction mixture was stirred for 24 to 36 h at 25 to 30° C. and the pH value was kept between 6.7 and 6.9 by addition of aqueous K₂CO₃ solution (20%). Then aqueous K₂CO₃ solution (20%) was added to the mixture to adjust pH to 7.6 to 8.0. MTBE (1044 kg) and celite (6.2 kg) were added to the mixture at 25 to 30° C. and the mixture was stirred for 0.5 hr. The obtained suspension was filtered, and the filter cake was washed with MTBE (64.5 kg) and water (84.2 kg). The layers were separated, and the organic layer (containing remaining ester) was put aside. The aqueous layer was extracted twice with MTBE (1044 kg and 519 kg, respectively), cooled to 10 to 20° C. and made acidic by dropwise addition of aqueous HCl (18.2%) to adjust pH to 1.4 to 1.6. The obtained suspension was cooled to −2 to +2° C. during 1 to 2 h, stirred for further 0.5 h and centrifuged. Water (421 kg) was added to the obtained solid and the suspension was stirred at 0 to 10° C. for 0.5 to 1.0 h. After centrifugation the wet solid was washed with cool water (211 kg) and dissolved in 2-propanol (IPA) (842 kg) at 70 to 75° C. The obtained suspension was filtered, the reactor was washed with hot IPA (421 kg) and the combined filtrate was concentrated to about 2 vol. at 40 to 50° C. The filtrate was warmed to 70 to 75° C., stirred for 0.5 h, cooled to −10 to ˜15° C. during 2 to 3 h, and stirred for 1 h. The obtained suspension was centrifuged to get a white solid that was dried in vacuo at 35 to 40° C. (JT) for 24 h and cooled to 20 to 30° C. under nitrogen atmosphere to give the product (105 kg) in crystalline form (I).

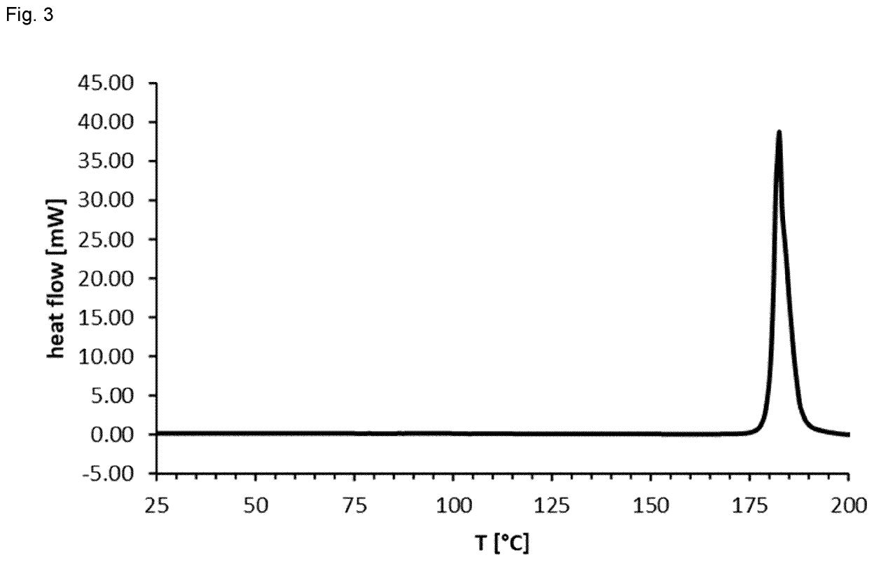

TABLE 2
Characterisation data for (R)-2-((tert-butoxycarbonyl)amino)-
3-(diethoxy-phosphoryl)propanoic acid in crystalline form (I)

| Technique | Data Summary | Remarks |
|---|---|---|
| XRPD | Crystalline | see FIG. 1 |
| 1H-NMR | δ = 12.7 (s, 1H), 7.07 (d, J = 8 Hz, 1H), | |
| (400 MHz, | 4.18 (m, 1H), 3.97 (m, 4H), 2.19 (dd, J1 = | |
| d⁶-DMSO) | 16 Hz, J2 = 8 Hz, 2H), 1.38 (m, 9H), 1.22 | |
| | (t, J = 8 Hz, 6H) | |
| Purity, | 99.5% | |
| HPLC (method 3) | rt = 17.9 min | |
| Chiral purity, | 99.9% | |
| chiral HPLC | rt = 5.3 min ((S)-isomer: rt = 6.3 min) | |
| (method 1) | | |
| GVS at 25° C. | Not hygroscopic | see FIG. 2 |
| DSC | Melting point: T = 180 ± 2° C. | see FIG. 3 |

### Example 5

### Racemisation (“recycling”) of ethyl(S)-2-((tert-butoxycarbonyl)amino)-3-(diethoxy-phosphoryl)propanoate (Side Product from Example 4)

A solution of ethyl(S)-2-((tert-butoxycarbonyl)amino)-3-(diethoxy-phosphoryl)propanoate (approximately 276 kg crude starting material) in MTBE (total weight of starting material and MTBE: 570 kg) was charged into the reactor and concentrated in vacuo to 1.0 to 1.5 vol. at 40 to 50° C. MTBE (1381 kg) was added and concentrated in vacuo to 1.0 to 1.5 vol. at 40 to 50° C. MTBE (1381 kg) was added again and concentrated in vacuo to 1.0 to 1.5 vol. at 40 to 50° C. MTBE (262 kg) was added to the mixture and stirred at 0 to 5° C. for 15 min. A 20% solution of NaOEt in EtOH (146 kg) was added dropwise within 1 to 2 h at 0 to 5° C. and the solution was stirred for additional 0.5 to 1 h at 0 to 5° C. AcOH (30.4 kg) was slowly added, the mixture was stirred for 0.5 h, MTBE (829 kg) and saturated, aqueous NaHCO₃ (829 kg) were added at 10 to 20° C., and the mixture was stirred for 0.5 h. The layers were separated, the organic layer was concentrated to dryness and the residue was dissolved in MTBE (140 kg) to obtain a solution of ethyl 2-((tert-butoxycarbonyl)amino)-3-(diethoxy-phosphoryl)propanoate (201 kg crude product) in MTBE that can be used in the enzymatic racemic resolution according to example 4.

### Example 6: Enzyme Screening

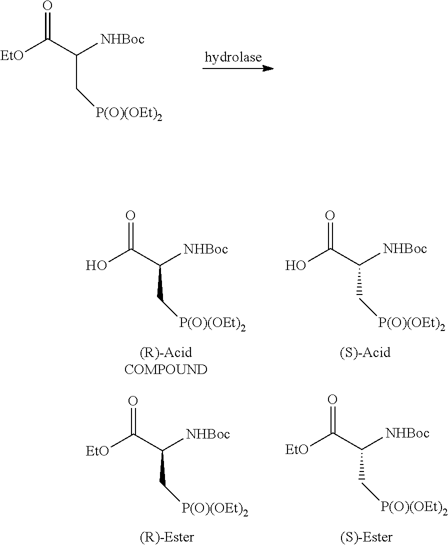

193 hydrolase enzymes were screened according to the following procedure: The respective enzyme (10 mg of lyophilized enzyme powder or 30 μL liquid enzyme or 10 mg immobilized enzyme) was dissolved in phosphate buffer (500 μL, 0.1 M, pH 7.0) and a solution of ethyl 2-((tert-butoxycarbonyl)amino)-3-(diethoxy-phosphoryl)propanoate (10 mg) in MTBE (37.5 μL) was added. The mixture was shaken at 210 rpm for 20 h at 25° C., adjusted to pH 1-2 with aqueous HCl (60 μL, 2 M) and extracted with MTBE (3×0.7 mL). The solution was concentrated in vacuo and the residue was taken up in EtOH (0.8 mL), filtered and analysed by chiral HPLC (method 2); retention times: (R)-acid: rt=9.1 min; (S)-acid: rt=23.8 min; (R)-ester: rt=13.7 min; (S)-ester: rt=29.8 min.

TABLE 3
Enzyme screening results (data are given for experiments resulting in an enantiomeric
excess (ee) of the acid of
at least 80%); the given amounts are % a/a based on chiral HPLC (method 2):

| | | | (R)-Acid | (R)-Ester | (S)-Acid | (S)-Ester |
|---|---|---|---|---|---|---|
| Supplier | Enzyme | Code | [% a/a] | [% a/a] | [% a/a] | [% a/a] |
| Almac | Microbial | AH002 | 8.8 | 37.9 | 0.7 | 52.6 |
| | hydrolase | | | | | |
| Almac | Microbial | AH008 | 25.4 | 15.6 | 0.3 | 58.6 |
| | hydrolase | | | | | |
| Almac | Microbial | AH012 | 39.1 | 0.9 | 1.3 | 58.7 |
| | hydrolase | | | | | |
| Almac | Microbial | AH016 | 19.8 | 20.9 | 0.2 | 59.0 |
| | hydrolase | | | | | |
| Almac | Microbial | AH017 | 39.0 | 0.1 | 3.6 | 57.3 |
| | hydrolase | | | | | |
| Almac | Microbial | AH018 | 35.7 | 2.2 | 0.5 | 61.6 |
| | hydrolase | | | | | |
| Almac | Microbial | AH019 | 39.0 | 0.1 | 1.1 | 59.7 |
| | hydrolase | | | | | |
| Almac | Microbial | AH022 | 38.2 | 0.3 | 0.8 | 60.7 |
| | hydrolase | | | | | |
| Almac | Microbial | AH023 | 45.0 | 1.4 | 1.7 | 51.9 |
| | hydrolase | | | | | |
| Almac | Microbial | AH025 | 38.0 | 0.6 | 0.9 | 60.4 |
| | hydrolase | | | | | |
| Almac | Microbial | AH027 | 37.5 | 1.5 | 0.9 | 60.2 |
| | hydrolase | | | | | |
| Almac | Microbial | AH028 | 34.2 | 0.7 | 0.2 | 65.0 |
| | hydrolase | | | | | |
| Almac | Microbial | AH032 | 36.4 | 0.1 | 0.4 | 63.1 |
| | hydrolase | | | | | |
| Almac | Microbial | AH034 | 43.9 | 0.5 | 0.4 | 55.2 |
| | hydrolase | | | | | |
| Almac | Microbial | AH035 | 33.1 | 8.8 | 0.5 | 57.6 |
| | hydrolase | | | | | |
| Almac | Microbial | AH036 | 34.6 | 9.1 | 0.5 | 55.8 |
| | hydrolase | | | | | |
| Almac | Microbial | AH037 | 8.9 | 39.3 | 0.3 | 51.5 |
| | hydrolase | | | | | |
| Almac | Microbial | AH041 | 42.4 | 0.7 | 1.7 | 55.2 |
| | hydrolase | | | | | |
| Almac | Microbial | AH042 | 21.2 | 20.4 | 0.5 | 57.9 |
| | hydrolase | | | | | |
| Almac | Microbial | AH044 | 41.6 | 0.1 | 1.2 | 57.1 |
| | hydrolase | | | | | |
| Almac | Microbial | AH045 | 26.8 | 12.1 | 1.2 | 59.9 |
| | hydrolase | | | | | |
| Almac | Microbial | AH047 | 40.3 | 0.5 | 1.3 | 58.0 |
| | hydrolase | | | | | |
| Almac | Microbial | AH048 | 28.5 | 16.8 | 0.5 | 54.2 |
| | hydrolase | | | | | |
| Almac | Microbial | AH051 | 34.7 | 9.6 | 0.8 | 54.8 |
| | hydrolase | | | | | |
| Almac | Microbial | AH052 | 17.1 | 26.7 | 0.2 | 55.9 |
| | hydrolase | | | | | |
| Almac | Microbial | AH055 | 37.2 | 0.7 | 0.9 | 61.3 |
| | hydrolase | | | | | |
| Almac | Microbial | AH056 | 20.5 | 21.3 | 1.2 | 57 |
| | hydrolase | | | | | |
| Almac | Microbial | AH057 | 44.6 | 0.1 | 3.2 | 52.1 |
| | hydrolase | | | | | |
| Almac | Microbial | AH059 | 38.8 | 0.0 | 0.8 | 60.4 |
| | hydrolase | | | | | |
| Almac | Microbial | AH060 | 9 | 38.3 | 0.3 | 52.3 |
| | hydrolase | | | | | |
| Almac | Microbial | AH061 | 39.1 | 0.1 | 0.9 | 60 |
| | hydrolase | | | | | |
| Almac | Microbial | AH062 | 42 | 0.9 | 2.5 | 54.5 |
| | hydrolase | | | | | |
| Almac | Microbial | CL055 | 41.1 | 1 | 1.2 | 56.7 |
| | hydrolase | | | | | |
| Almac | Microbial | CL067 | 40.8 | 0.5 | 0.7 | 58.1 |
| | hydrolase | | | | | |
| Amano | Protease from | Protease M | 38.2 | 1.1 | 1.6 | 59.1 |
| | Aspergillus | | | | | |
| | Oryzae | | | | | |
| Eucodis | Microbial | EU62 | 30.8 | 8.3 | 0.6 | 60.2 |
| | hydrolase | | | | | |
| DSM | Pig liver | DSM-A1 | 41.1 | 0.1 | 4.6 | 54 |
| Innosyn | esterase | | | | | |
| | variant | | | | | |
| DSM | Pig liver | DSM-A2 | 19.5 | 24.7 | 1.1 | 54.7 |
| Innosyn | esterase | | | | | |
| | variant | | | | | |
| DSM | Pig liver | DSM-A3 | 35.5 | 4.8 | 0.6 | 59.1 |
| Innosyn | esterase | | | | | |
| | variant | | | | | |
| DSM | Pig liver | DSM-A6 | 33.2 | 7.5 | 1.1 | 58.1 |
| Innosyn | esterase | | | | | |
| | variant | | | | | |
| DSM | Pig liver | DSM-B1 | 38.9 | 1.2 | 0.8 | 59 |
| Innosyn | esterase | | | | | |
| | variant | | | | | |
| DSM | Pig liver | DSM-B2 | 39.1 | 0.7 | 0.9 | 59.3 |
| Innosyn | esterase | | | | | |
| | variant | | | | | |
| DSM | Pig liver | DSM-B3 | 29.7 | 11.3 | 1.3 | 57.7 |
| Innosyn | esterase | | | | | |
| | variant | | | | | |
| DSM | Pig liver | DSM-B6 | 9.8 | 37.7 | 1 | 51.5 |
| Innosyn | esterase | | | | | |
| | variant | | | | | |
| DSM | Pig liver | DSM-C1 | 33.6 | 6.9 | 0.4 | 59.2 |
| Innosyn | esterase | | | | | |
| | variant | | | | | |
| DSM | Pig liver | DSM-C2 | 28 | 11.7 | 1.17 | 59.1 |
| Innosyn | esterase | | | | | |
| | variant | | | | | |
| DSM | Pig liver | DSM-C3 | 39.1 | 0.14 | 1.08 | 59.7 |
| Innosyn | esterase | | | | | |
| | variant | | | | | |
| DSM | Pig liver | DSM-D2 | 9.7 | 35.7 | 0.4 | 54.2 |
| Innosyn | esterase | | | | | |
| | variant | | | | | |
| DSM | Pig liver | DSM-D3 | 14.2 | 29.4 | 0.1 | 56.3 |
| Innosyn | esterase | | | | | |
| | variant | | | | | |

### Example 7: Optimization of Enzyme Loading and Use of Co-Solvent

### Condition A:

The respective enzyme (5 mg of lyophilized enzyme powder) was dissolved in phosphate buffer (250 μL, 0.1 M, pH 7.0) and a solution of ethyl 2-((tert-butoxycarbonyl)amino)-3-(diethoxy-phosphoryl)propanoate (5 mg) in MTBE (12.5 μL) was added. The mixture was shaken at 250 rpm for 20 h at 30° C. and further treated according to the workup conditions.

### Condition B:

A stock solution of the respective enzyme (0.5 mg) in phosphate buffer (119 μL, 0.1 M, pH 7.0) was diluted with phosphate buffer (119 μL, 0.1 M, pH 7.0) and a solution of ethyl 2-((tert-butoxycarbonyl)amino)-3-(diethoxy-phosphoryl)propanoate (5 mg) in MTBE (12.5 μL) was added. The mixture was shaken at 250 rpm for 20 h at 30° C. and further treated according to the workup conditions.

### Condition C:

A stock solution of the respective enzyme (0.5 mg) in phosphate buffer (119 μL, 0.1 M, pH 7.0) was treated with a solution of ethyl 2-((tert-butoxycarbonyl)amino)-3-(diethoxy-phosphoryl)propanoate (5 mg) in phosphate buffer (131 μL, 0.1 M, pH 7.0). The mixture was shaken at 250 rpm for 20 h at 30° C. and further treated according to the workup conditions.

### Condition D:

A solution of ethyl 2-((tert-butoxycarbonyl)amino)-3-(diethoxy-phosphoryl)propanoate (25 mg/mL solvent) and the respective enzyme (1% per weight relative to ester) in phosphate buffer (0.1 M, pH 7.0) and MTBE (5% total volume) was shaken at 250 rpm for 18 h at 30° C. and further treated according to the workup conditions.

### Condition E:

A solution of ethyl 2-((tert-butoxycarbonyl)amino)-3-(diethoxy-phosphoryl)propanoate (100 mg/mL solvent) and the respective enzyme (1% per weight relative to ester) in phosphate buffer (0.1 M, pH 7.0) and MTBE (5% total volume) was shaken at 250 rpm for 18 h at 30° C. and further treated according to the workup conditions.

### Workup Conditions:

To each vial was added MTBE (1 mL), followed by aqueous HCl (20 μL, 2 M). The mixture was shaken at 25° C. at 200 rpm for 30 minutes before centrifuging to obtain distinct layers. A portion of the organic layer (0.8 mL) was removed and dried through a MgSO₄ pipette into clean Eppendorf vials. The MTBE was then removed in a Genevac rotary vacuum pump, taken up in EtOH (200 μL) and analysed by chiral HPLC (method 2); retention times: (R)-acid: rt=9.1 min; (S)-acid: rt=23.8 min; (R)-ester: rt=13.7 min; (S)-ester: rt=29.8 min.

TABLE 4
Enzyme screening results for experiments under Condition
A (100% enzyme, 5% MTBE); the given amounts are % a/a
based on chiral HPLC (method 2); supplier and enzyme
specifics for the given codes are contained in table 3:

| | (R)-Acid | (R)-Ester | (S)-Acid | (S)-Ester |
|---|---|---|---|---|
| Code | [% a/a] | [% a/a] | [% a/a] | [% a/a] |
| AH012 | 30.2 | 10.7 | 0.4 | 58.7 |
| AH016 | 32.7 | 9.3 | 0.4 | 57.6 |
| AH017 | 37.2 | 4.0 | 1.2 | 57.6 |
| AH018 | 42.0 | 0.1 | 3.2 | 54.7 |
| AH022 | 41.1 | 0.0 | 3.4 | 55.5 |
| AH023 | 42.8 | 0.2 | 4.0 | 53.0 |
| AH025 | 40.4 | 0.3 | 3.5 | 55.8 |
| AH027 | 39.8 | 0.1 | 0.3 | 59.5 |
| AH028 | 30.8 | 10.8 | 0.3 | 58.2 |
| AH032 | 39.1 | 0.5 | 0.3 | 60.1 |
| AH034 | 40.1 | 0.2 | 1.6 | 58.0 |
| AH035 | 22.1 | 21.9 | 0.2 | 55.8 |
| AH036 | 1.3 | 48.0 | 0.2 | 50.4 |
| AH041 | 43.6 | 0.1 | 9.4 | 46.9 |
| AH042 | 22.8 | 17.0 | 0.2 | 60.0 |
| AH044 | 39.7 | 0.0 | 0.3 | 60.0 |
| AH047 | 38.9 | 0.4 | 0.8 | 60.0 |
| AH048 | 26.5 | 18.8 | 0.2 | 54.5 |
| AH051 | 40.9 | 2.5 | 12.0 | 44.6 |
| AH052 | 10.0 | 36.8 | 0.2 | 53.0 |
| AH055 | 43.4 | 0.1 | 20.5 | 36.0 |
| AH059 | 40.0 | 0.0 | 1.3 | 58.6 |
| AH061 | 34.6 | 0.2 | 0.7 | 64.5 |
| Protease M | 39.1 | 0.9 | 3.2 | 56.8 |

(R)-Acid, (S)-Acid, (R)-Ester and (S)-Ester: see example 6

TABLE 5
Enzyme screening results for experiments under Condition
B (10% enzyme, 5% MTBE); the given amounts are % a/a
based on chiral HPLC (method 2); supplier and enzyme
specifics for the given codes are contained in table 3:

| | (R)-Acid | (R)-Ester | (S)-Acid | (S)-Ester |
|---|---|---|---|---|
| Code | [% a/a] | [% a/a] | [% a/a] | [% a/a] |
| AH012 | 1.9 | 47.1 | 0.2 | 50.8 |
| AH016 | 5.6 | 42.4 | 0.3 | 51.8 |
| AH017 | 12.6 | 32.5 | 0.4 | 54.6 |
| AH018 | 36.8 | 0.6 | 0.7 | 61.9 |
| AH022 | 36.4 | 0.2 | 0.9 | 62.5 |
| AH023 | 31.8 | 3.3 | 0.2 | 64.7 |
| AH025 | 29.9 | 10.6 | 1.0 | 58.5 |
| AH027 | 34.2 | 3.3 | 0.3 | 62.1 |
| AH028 | 8.9 | 37.9 | 0.5 | 52.8 |
| AH032 | 2.4 | 45.2 | 0.4 | 52.0 |
| AH034 | 35.7 | 1.0 | 0.3 | 62.9 |
| AH035 | 4.3 | 43.0 | 0.3 | 52.4 |
| AH036 | 1.3 | 46.5 | 0.5 | 51.7 |
| AH041 | 37.8 | 0.0 | 2.1 | 60.1 |
| AH042 | 3.9 | 40.0 | 1.7 | 54.4 |
| AH044 | 35.8 | 1.6 | 0.3 | 62.3 |
| AH047 | 35.9 | 1.2 | 0.5 | 62.4 |
| AH048 | 1.8 | 47.6 | 0.2 | 50.3 |
| AH051 | 39.3 | 0.2 | 4.5 | 55.9 |
| AH052 | 0.2 | 49.6 | 0.3 | 49.8 |
| AH055 | 35.8 | 1.3 | 0.3 | 62.6 |
| AH059 | 38.2 | 0.0 | 6.7 | 55.1 |
| AH061 | 34.8 | 0.7 | 0.3 | 64.3 |
| Protease M | 37.0 | 0.1 | 0.4 | 62.6 |

(R)-Acid, (S)-Acid, (R)-Ester and (S)-Ester: see example 6

TABLE 6
Enzyme screening results for experiments under Condition
C (10% enzyme, no MTBE); the given amounts are % a/a
based on chiral HPLC (method 2); supplier and enzyme
specifics for the given codes are contained in table 3:

| | (R)-Acid | (R)-Ester | (S)-Acid | (S)-Ester |
|---|---|---|---|---|
| Code | [% a/a] | [% a/a] | [% a/a] | [% a/a] |
| AH012 | 2.0 | 43.9 | 1.1 | 53.1 |
| AH016 | 6.3 | 41.2 | 0.4 | 52.1 |
| AH017 | 12.0 | 34.6 | 0.3 | 53.1 |
| AH018 | 37.4 | 0.0 | 0.7 | 61.9 |
| AH022 | 37.1 | 0.1 | 0.6 | 62.2 |
| AH023 | 36.6 | 0.1 | 0.6 | 62.8 |
| AH025 | 30.0 | 5.6 | 0.8 | 63.6 |
| AH027 | 32.4 | 4.4 | 0.2 | 63.1 |
| AH028 | 6.8 | 40.5 | 0.4 | 52.4 |
| AH032 | 4.5 | 44.0 | 0.8 | 50.7 |
| AH034 | 36.0 | 0.1 | 0.4 | 63.6 |
| AH035 | 4.9 | 45.4 | 0.2 | 49.8 |
| AH036 | 1.8 | 46.6 | 0.3 | 51.0 |
| AH041 | 36.3 | 0.1 | 2.4 | 61.2 |
| AH042 | 4.4 | 44.2 | 0.4 | 51.0 |
| AH044 | 36.8 | 0.0 | 0.3 | 62.8 |
| AH047 | 37.0 | 0.0 | 0.5 | 62.4 |
| AH048 | 1.4 | 46.8 | 0.4 | 51.5 |
| AH051 | 39.6 | 0.0 | 7.0 | 53.4 |
| AH052 | 4.6 | 42.4 | 2.0 | 51.0 |
| AH055 | 38.6 | 0.1 | 10.2 | 51.1 |
| AH059 | 37.2 | 0.8 | 0.5 | 62.0 |
| AH061 | 35.2 | 3.1 | 0.5 | 61.2 |
| Protease M | 36.7 | 0.0 | 0.7 | 62.6 |

(R)-Acid, (S)-Acid, (R)-Ester and (S)-Ester: see example 6

TABLE 7
Enzyme screening results for experiments under Condition D
(25 mg/mL substrate, 1% enzyme, 5% MTBE); the given amounts
are % a/a based on chiral HPLC (method 2); supplier and enzyme
specifics for the given codes are contained in table 3:

| | (R)-Acid | (R)-Ester | (S)-Acid | (S)-Ester |
|---|---|---|---|---|
| Code | [% a/a] | [% a/a] | [% a/a] | [% a/a] |
| AH018 | 34.9 | 0.7 | 0.3 | 64.1 |
| AH022 | 34.4 | 2.1 | 0.29 | 63.2 |
| AH023 | 36.4 | 0.2 | 0.19 | 63.2 |
| AH027 | 6.0 | 40.0 | 0.26 | 53.7 |
| AH034 | 33.7 | 1.9 | 0.26 | 64.2 |
| AH044 | 24.9 | 15.4 | 0.2 | 59.5 |
| AH047 | 14.4 | 30.5 | 0.26 | 54.8 |
| Protease M | 35.5 | 0.4 | 0.24 | 63.9 |

(R)-Acid, (S)-Acid, (R)-Ester and (S)-Ester: see example 6

TABLE 8
Enzyme screening results for experiments under Condition E (100
mg/mL substrate, 1% enzyme, 5% MTBE); the given amounts are
% a/a based on chiral HPLC (method 2); supplier and enzyme
specifics for the given codes are contained in table 3:

| | (R)-Acid | (R)-Ester | (S)-Acid | (S)-Ester |
|---|---|---|---|---|
| Code | [% a/a] | [% a/a] | [% a/a] | [% a/a] |
| AH018 | 9.4 | 36.7 | 0.06 | 53.7 |
| AH022 | 9.6 | 36.7 | 0.1 | 53.6 |
| AH023 | 17.4 | 26.2 | 0.01 | 56.3 |
| AH027 | 2.4 | 45.3 | 0.1 | 52.2 |
| AH034 | 12.9 | 32.2 | 0.1 | 54.8 |
| AH044 | 5.1 | 42.2 | 0.1 | 52.5 |
| AH047 | 3.0 | 44.5 | 0.1 | 52.3 |
| Protease M | 19.2 | 24.3 | 0.06 | 56.4 |

(R)-Acid, (S)-Acid, (R)-Ester and (S)-Ester: see example 6

### Example 8: Synthesis of (R)-2-((tert-butoxycarbonyl)amino)-3-(diethoxyphosphoryl) propanoic acid (1 g Scale)

To a 50 mL 3-necked RBF was added ethyl 2-((tert-butoxycarbonyl)amino)-3-(diethoxy-phosphoryl)propanoate (1 g, 2.83 mmol) in MTBE (0.5 mL+0.1 mL MTBE as line wash). To this solution was added phosphate buffer (0.1 M, pH 7.0, 9 mL) and the biphasic mixture was heated to 30° C. under stirring. To this biphasic solution was added Protease M from Amano (10 mg in 0.5 mL phosphate buffer) and the solution was stirred for 18 hours. The reaction pH was maintained between pH 6.8 and 7.0 with addition of 20% K₂CO₃ (aq). The reaction was adjusted to pH 8.0 and extracted with MTBE (2×10 mL). The aqueous portion was recharged to the reaction vessel and adjusted to pH 1.5 with 2M HCl, at which point a rapid precipitation occurred. The suspension was cooled to 0° C. in an ice-water bath and stirred for 30 minutes at this temperature. The suspension was filtered, washed successively with 0.1 M phosphate buffer (pH 1.5, 2 mL) and 0.1 M HCl (4 mL), and pulled to dryness over 1 hour. (R)-2-((tert-butoxycarbonyl)amino)-3-(diethoxyphosphoryl)propanoic acid was obtained as white solid (420 mg, 45.6%) in 99% ee (chiral HPLC (method 3); retention times: (R)-acid: rt =5.7; (S)-acid: rt=6.6 min; (R)-ester: rt=25.3 min; (S)-ester: rt=28.1 min.

### Example 9: Synthesis of (R)-2-((tert-butoxycarbonyl)amino)-3-(diethoxyphosphoryl) propanoic acid

Water (3.6 L; 6.6 vol) was added to a 10 L reactor and vigorously agitated. Potassium phosphate monobasic (49.0 g; 360 mmol) was added and the pH of the reactor contents was adjusted to pH 6.8 (+0.2) with 20% (w/v) aqueous potassium carbonate solution. Ethyl 2-((tert-butoxycarbonyl)amino)-3-(diethoxyphosphoryl)propanoate (1.55 mmol) was dissolved in MTBE (360 mL) in a 1 L glass bottle and added to the reactor. The glass bottle was washed with MTBE (90 mL) and the solution was added to the reactor. Protease M from Amano (6.0 g) was dissolved in purified water (55 mL) in a 100 ml bottle and added to the reactor in a single portion. The bottle was washed with purified water (27.5 mL) and the solution was added to reactor. The reaction mixture was stirred for 8 hours at 28±2° C. and maintained at pH 6.8 by addition of 20% (w/v) aqueous potassium carbonate solution. The external heating was turned off and the mixture was stirred for additional 15 hours. The pH was adjusted to 7.8±0.2 with 20% (w/v) aqueous potassium carbonate solution. Celite (12.0 g) and MTBE (4.1 L) were charged to the reactor and the reactor content was filtered through a Buchner funnel after 10 min. The biphasic mixture was recharged to the reactor and layers were allowed to separate for a minimum of 5 min. The organic layer (containing remaining ester) was stored in a 25 L HDPE drum and the aqueous layer was recharged to reactor. MTBE (4.1 L) was added, the mixture was agitated for 5 min., the layers were separated and the aqueous layer was discharged. The organic layer was combined with the earlier organic layer. The aqueous layer was recharged to reactor, MTBE (2.05 L) was added, the mixture was agitated for 5 min., the layers were separated and the aqueous layer was discharged. The organic layer was combined with the earlier organic layers. The aqueous layer was recharged to the reactor and adjusted to pH 1.5 via dropwise addition of 18.5% aqueous HCl. The reactor contents were cooled to 3° C. +2° C., stirred gently for 30 min and filtered through a buchner funnel. The filter cake was washed with chilled aqueous HCl solution (0.1 M, 820 mL) and pulled dry on funnel for 1 hour. The cake was further dried to constant weight in vacuum at 50° C. to give (R)-2-((tert-butoxycarbonyl)amino)-3-(diethoxyphosphoryl)propanoic acid in 46% yield, 98% purity (NMR) and 99.9% ee (chiral HPLC (method 3)).

1-15. (canceled)

16. A process for the manufacturing of a compound of formula (I), or of a salt thereof, 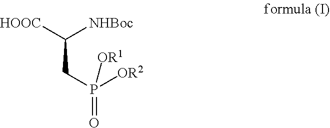
said process comprising the step of reacting a compound of formula (II) 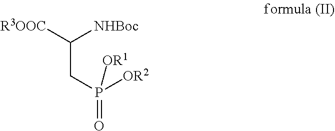
wherein
R¹ and R² represent independently from each other (C₁₋₄)alkyl; and
R³ represents methyl, ethyl or n-propyl;
with a hydrolase to give the compound of formula (I) with an enantiomeric excess (ee) of at least 70%.

17. A process according to claim 16, wherein the hydrolase is selected from AH002, AH008, AH012, AH016, AH017, AH018, AH019, AH022, AH023, AH025, AH027, AH028, AH032, AH034, AH035, AH036, AH037, AH041, AH042, AH044, AH045, AH047, AH048, AH051, AH052, AH055, AH056, AH057, AH059, AH060, AH061, AH062, CL055, CL067, Protease M, EU62, DSM-A1, DSM-A2, DSM-A3, DSM-A6, DSM-B1, DSM-B2, DSM-B3, DSM-B6, DSM-C1, DSM-C2, DSM-C3, DSM-D2, and DSM-D3.

18. A process according to claim 16, wherein the hydrolase is selected from AH018, AH022, AH023, AH027, AH034, AH044, AH047, and Protease M.

19. A process according to any one of claim 16, wherein the hydrolase is Protease M.

20. A process according to claim 16, wherein the process gives the compound of formula (I) with an enantiomeric excess (ee) of at least 96%.

21. A process according to claim 16, wherein R¹ and R² both represent ethyl.

22. A process according to claim 16, wherein R³ represents methyl or ethyl.

23. A process according to claim 16, wherein the reaction (enzymatic resolution) is conducted in a mixture of water and an organic solvent selected from MTBE, DMSO, toluene and any mixture thereof.

24. A process according to claim 16, wherein the reaction (enzymatic resolution) is conducted at a pH value between 5.0 and 8.0.

25. A process according to claim 16, wherein the reaction (enzymatic resolution) is conducted with an enzyme loading of between 0.2% and 10%.

26. A process according to claim 16, wherein the process further comprises the steps of reacting a compound of formula (V) 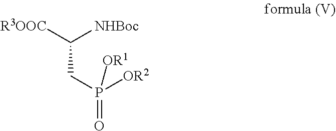
with a base to give a compound of formula (II) 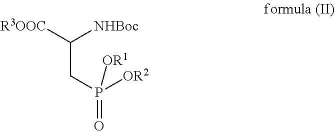
wherein
R¹ and R² represent independently from each other (C₁₋₄)alkyl; and
R³ represents methyl, ethyl or n-propyl;
and wherein the compound of formula (V) has an enantiomeric excess (ee) of at least 80% and the compound of formula (II) has an enantiomeric excess (ee) of less than 10%.

27. A process for the manufacturing of a compound of formula (I), or of a salt thereof, 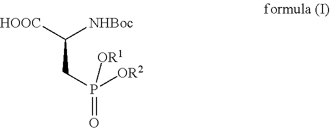
said process comprising the step of reacting a compound of formula (II) 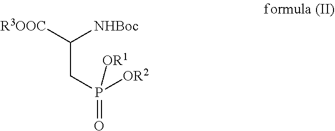
wherein
R¹ and R² represent independently from each other (C₁₋₄)alkyl; and
R³ represents methyl, ethyl or n-propyl;
with a hydrolase, wherein the hydrolase is selected from AH002, AH008, AH012, AH016, AH017, AH018, AH019, AH022, AH023, AH025, AH027, AH028, AH032, AH034, AH035, AH036, AH037, AH041, AH042, AH044, AH045, AH047, AH048, AH051, AH052, AH055, AH056, AH057, AH059, AH060, AH061, AH062, CL055, CL067, Protease M, EU62, DSM-A1, DSM-A2, DSM-A3, DSM-A6, DSM-B1, DSM-B2, DSM-B3, DSM-B6, DSM-C1, DSM-C₂, DSM-C3, DSM-D2, and DSM-D3.

28. A process according to claim 27, wherein the hydrolase is selected from AH018, AH022, AH023, AH027, AH034, AH044, AH047, and Protease M.

29. A process according to claim 27, wherein the hydrolase is Protease M.

30. A process according to claim 27, wherein the process gives the compound of formula (I) with an enantiomeric excess (ee) of at least 96%.

31. A process according to claim 27, wherein R¹ and R² both represent ethyl.

32. A process according to claim 27, wherein R³ represents methyl or ethyl.

33. A process according to claim 27, wherein the reaction (enzymatic resolution) is conducted in a mixture of water and an organic solvent selected from MTBE, DMSO, toluene and any mixture thereof.

34. A process according to claim 27, wherein the reaction (enzymatic resolution) is conducted at a pH value between 5.0 and 8.0.

35. A process according to claim 27, wherein the reaction (enzymatic resolution) is conducted with an enzyme loading of between 0.2% and 10%.

36. A process according to claim 27, wherein the process further comprises the steps of reacting a compound of formula (V) 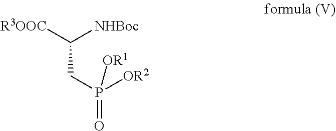
with a base to give a compound of formula (II) 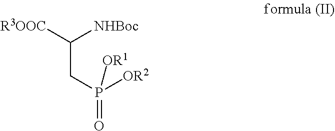
wherein
R¹ and R² represent independently from each other (C₁₋₄)alkyl; and
R³ represents methyl, ethyl or n-propyl;
and wherein the compound of formula (V) has an enantiomeric excess (ee) of at least 80% and the compound of formula (II) has an enantiomeric excess (ee) of less than 10%.

37. A process for the manufacturing of selatogrel 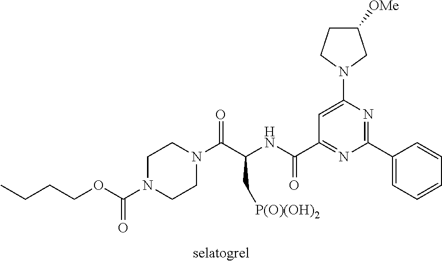
wherein the process comprises the step of reacting a compound of formula (II) 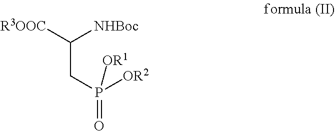
wherein
R¹ and R² represent independently from each other (C₁₋₄)alkyl; and
R³ represents methyl, ethyl or n-propyl;
with a hydrolase to give a compound of formula (I), or a salt thereof, with an enantiomeric excess (ee) of at least 70%

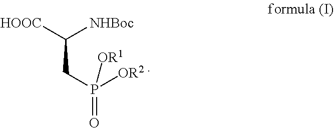

38. A process according to claim 37, wherein the hydrolase is selected from AH018, AH022, AH023, AH027, AH034, AH044, AH047, and Protease M.

39. A process according to claim 37, wherein the hydrolase is Protease M.

40. A process according to claim 37, wherein the process gives the compound of formula (I) with an enantiomeric excess (ee) of at least 96%.

41. A process according to claim 37, wherein R¹ and R² both represent ethyl.

42. A process according to claim 37, wherein R³ represents methyl or ethyl.

43. A process according to claim 37, wherein the reaction (enzymatic resolution) is conducted in a mixture of water and an organic solvent selected from MTBE, DMSO, toluene and any mixture thereof.

44. A process according to claim 37, wherein the reaction (enzymatic resolution) is conducted at a pH value between 5.0 and 8.0.

45. A process according to claim 37, wherein the reaction (enzymatic resolution) is conducted with an enzyme loading of between 0.2% and 10%.

46. A process according to claim 37, wherein the process further comprises the steps of reacting a compound of formula (V) 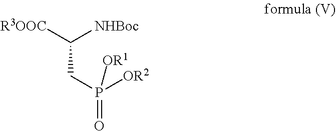
with a base to give a compound of formula (II) 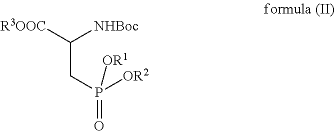
wherein
R¹ and R² represent independently from each other (C₁₋₄)alkyl; and
R³ represents methyl, ethyl or n-propyl;
and wherein the compound of formula (V) has an enantiomeric excess (ee) of at least 80% and the compound of formula (II) has an enantiomeric excess (ee) of less than 10%.

47. A crystalline form of (R)-2-((tert-butoxycarbonyl)amino)-3-(diethoxyphosphoryl)propanoic acid, characterized by the presence of peaks in the X-ray powder diffraction diagram at the following angles of refraction 2θ: 9.8°, 10.3°, and 17.5°.

48. A crystalline form of (R)-2-((tert-butoxycarbonyl)amino)-3-(diethoxyphosphoryl)propanoic acid according to claim 47, characterized by an endothermic peak at about 180° C. as measured by DSC.
